(19) United States
(12) Patent Application Publication
(10) Pub. No.: US 2025/0011423 A1
(43) Pub. Date: Jan. 9, 2025

(54) METHODS TO REVERSE TREML1-INDUCED IMMUNE SUPPRESSION

(71) Applicants: Frank Wen-Chi LEE, Orlando, FL (US); Ascendo Biotechnology, Inc., Grand Cayman (KY)

(72) Inventors: Yen-Ta Lu, Taipei (TW); Chia-Ming Chang, Taipei (TW); Yi-Chen Chen, Taipei (TW); I-Fang Tsai, Taipei (TW)

(73) Assignee: Ascendo Biotechnology, Inc., Grand Cayman (KY)

(21) Appl. No.: 18/710,549

(22) PCT Filed: Nov. 15, 2022
(86) PCT No.: PCT/US2022/079865

§ 371 Date: May 15, 2024

Related U.S. Application Data
(60) Provisional application No. 63/279,670, filed on Nov. 15, 2021.

(51) Int. Cl. C07K 16/28; A61K 39/00; A61P 35/00; G01N 33/574

(52) U.S. Cl. CPC C07K 16/2803; A61P 35/00; G01N 33/57484; A61K 2039/505; C07K 2317/24; C07K 2317/31; G01N 2333/70503

(57) ABSTRACT

Soluble TREML1 is involved in many inflammatory diseases and plays an important role in immune suppression. Soluble TREML1 can bind the l-domain of CD11b and induce an immune suppressive phenotype. Thus, soluble TREML1 may be a good target for treating inflammatory diseases. The present invention relates to antibodies that can reduce soluble TREML1 binding to CD11b+ immune cells and their uses for reversing TREML14 induced immune suppression. Anti-TREML1 antibodies provide a novel and potential therapy for these disease conditions such as cancer.

### CROSS REFERENCE TO RELATED APPLICATIONS

This application is a U.S. National Phase of International Application No. PCT/US2022/079865, filed Nov. 15, 2022, which claims the benefit of U.S. Provisional Patent Application 63/279,670, filed on Nov. 15, 2021, which are hereby incorporated by reference in their entirety.

### SEQUENCE LISTING INFORMATION

The Sequence Listing associated with this application is provided in XML format in lieu of a paper copy and is hereby incorporated by reference into the specification. The name of the XML file containing the Sequence Listing is A263-1005PCT.xml. The XML file is 51,147 bytes, was created on Oct. 20, 2022, and is being submitted electronically via PatentCenter.

### TECHNICAL FIELD

The present disclosure describes antibodies, compositions comprising antibodies, and methods of using the antibodies for inhibiting or reversing immune suppression.

### BACKGROUND

TREM-like transcript-1 (TREML1; TLT-1) is a member of the TREM family. TREML1 consists of a single V-set immunoglobulin (Ig) domain, a stalk region that contains a charged residue, a transmembrane domain, and a cytoplasmic tail. TREML1 is exclusively found in platelets in the peripheral blood of humans. Upon activation, TREML1 is quickly exposed to the membrane of platelets, and the extracellular domain of TREML1 (TREML1 ECD) is subsequently cleaved, leading to the release of a soluble TREML1. Studies have shown that patients with sepsis, in contrast to healthy individuals, have increased levels of soluble TREML1 in the plasma. Patients who have died from sepsis had sustained high levels of soluble TREML1 in the plasma, whereas those who survived showed declines in soluble TREML1 during this same period. Indicators of immune suppression observed in patients with sepsis include lymphocyte abnormalities, monocytic deactivation with diminished human leukocyte antigen-DR (HLA-DR) surface expression, and low TNF-α production under ex vivo stimulation. Sustained reductions in monocyte HLA-DR expression indicate a high risk for nosocomial infection and death in patients with sepsis. Recently, elevated program death ligand-1 (PD-L1) expression on monocytes in patients with septic shock was observed and associated with increased secondary nosocomial infections and mortality. Previously, it was reported that soluble TREML1 directly binds to immune cells and triggers cells to express immunosuppressive phenotypes, such as down-regulation of HLA-DR and up-regulation of PD-L1 (WO2016197975A1). In other diseases, such as acute respiratory distress syndrome, acute coronary syndrome, and coronary artery disease, high soluble TREML1 plasma concentrations have also been associated with a negative outcome. These results suggest that soluble TREML1 plays an important role in inflammation-related diseases. Thus, soluble TREML1 can serve as a biomarker and therapeutic target for inflammation-related diseases.

Macrophage-1 antigen (Mac-1, integrin αMb2, CD11b/CD18) is mainly expressed on the surface of innate immune cells (including monocytes, neutrophils, NK cells, etc.) and some populations of B cells (Proc. Natl. Acad. Sci. U.S.A. 2008 Apr. 1; 105(13):5195-200) and T cells (J. Immunol. 2001 Jan. 15; 166(2):900-7). Mac-1 is a heterodimeric glycoprotein comprising non-covalently linked integrin αM (CD11b, CR3A, ITGAM) and integrin β2 (CD18, ITGB2). CD11b is a transmembrane protein with a large extracellular domain and a short cytoplasmic tail. Its extracellular domain comprises an I-domain, a β-propeller domain, a thigh domain, a calf-1 domain, and a calf-2 domain. The I-domain of CD11b has around 179 amino acids inserted into the β-propeller domain. This I-domain is responsible for binding to promiscuous ligands (e.g., iC3b, fibrinogen, ICAM-1, CD40L, etc.) and participates in cell adhesion, migration, chemotaxis, and phagocytosis, and regulates inflammatory responses of immune cells.

There is a need to use new targets for developing and treating inflammation-related diseases.

### SUMMARY

This Summary is provided to introduce a selection of concepts in a simplified form that is further described below in the Detailed Description. This Summary is not intended to identify all key features or essential features of the claimed subject matter, nor is it intended to be used alone as an aid in determining the scope of the claimed subject matter.

The present disclosure is based on the surprising finding that TREML1 ECD can bind to the I-domain of CD11 b. As a result of TREML1 ECD binding to CD11 b+ immune cells, TREML1 ECD can induce immune suppression. Anti-CD11 b antibodies can reverse TREML1 ECD-induced immune suppression. Anti-TREML1 antibody treatments also reduce TREML1 ECD binding to CD11b+ immune cells. Thus, anti-TREML1 antibodies can be used to treat immune suppressive disorders, such as cancer.

The present disclosure describes agents including antibodies that bind TREML1, These antibodies can suppress tumor growth by preventing the binding of TREML1 ECD to the CD11 b+ immune cells. The present disclosure also describes compositions and pharmaceutical compositions comprising the agents described herein including antibodies that bind TREML1. In embodiments, the antibodies bind TREML1 ECD.

Moreover, the present disclosure describes methods of reversing immune suppression using agents including antibodies described herein.

Further, the present disclosure describes methods of using the agents, such as the antibodies described herein, to treat various diseases and conditions associated with immune suppression including cancer.

Furthermore, the present disclosure describes methods for diagnosing cancer or detecting the presence of a malignant tumor in a subject and/or for determining whether a subject would be responsive to various cancer therapies including immunotherapy and other forms of cancer treatments.

### BRIEF DESCRIPTION OF THE DRAWINGS

FIG. 1 shows that TREML1 ECD can bind to CD11b of human monocyte and neutrophil and that CD11 b specific antibody can compete with TREML1 ECD binding to CD11b+ immune cells (Monocyte and neutrophils). White blood cells (WBCs) were incubated with 10 μg/ml of human TREML1 ECD and 10 μg/ml of anti-CD11 b antibody (ICRF44). After treatment, cells were washed with staining buffer and stained with Alexa Fluor 488-conjugated anti-human TREML1 antibody (FAB2394G, R&D systems). Monocytes and neutrophils were analyzed by flow cytometry. MFI is mean fluorescence intensity.

FIG. 2 shows that TREML1 ECD binds to the I-domain of CD11b in a concentration-dependent manner.

FIG. 3 shows that the anti-CD11 b antibody can reverse TREML1-induced immune suppression. Monocytes were incubated with human TREML1 ECD (10 μg/ml) in the presence of either 10 μg/ml of isotype control IgG (MOPC21) or 10 μg/ml of anti-CD11b antibody (ICRF44) for three days. Cells were harvested and analyzed by flow cytometry at the indicated time points. The HLA-DR and PD-L1 expression of isotype control IgG-treated or anti-CD11 b antibody-treated monocytes are presented relative to the HLA-DR and PD-L1 expression of monocytes with mock (control) treatment at the same time point.

FIG. 4 shows that the anti-TREML1 antibody binds to TREML1 ECD in a concentration-dependent manner.

FIG. 5 shows that anti-TREML1 antibody can bind to HEK293 cells expressing human or mouse TREML1 on the cell surface. The HEK293/human TREML1 or HEK293/mouse TREML1 were treated with 10 μg/ml anti-TREML1 antibodies or isotype control IgG (MOPC21). The cells were then incubated at 37° C. for 30 min. Cells were then treated with APC conjugated anti-mouse IgG antibodies and analyzed by flow cytometry.

FIGS. 6A and 6B show that anti-TREML1 antibody can block TREML1 ECD binding to CD11 b+ immune cells (monocytes and neutrophils). (A) WBCs were incubated with various concentrations of human TREML1 ECD and 10 μg/ml of anti-TREML1 antibody (26A6). After treatment, cells were washed with staining buffer and stained with Alexa Fluor 488-conjugated anti-human TREML1 antibody (FAB2394G, R&D systems). Monocytes and neutrophils were analyzed by flow cytometry. (B) WBCs were incubated with 10 μg/ml of human TREML1 ECD and 10 μg/ml of anti-TREML1 antibody (18D8, 23F10, 27D3, 28A9, and 29F4). After treatment, cells were washed with staining buffer and stained with Alexa Fluor 488-conjugated anti-human TREML1 antibody (FAB2394G, R&D systems). Monocytes and neutrophils were analyzed by flow cytometry.

FIG. 7 shows that anti-TREML1 antibody can reverse TREML1-induced immune suppression. Monocytes were incubated with human TREML1 ECD (1, 5, 10 μg/ml) in the presence of either 10 μg/ml of isotype control IgG (MOPC21) or 10 μg/ml of anti-TREML1 antibody (26A6) for 24 hours (hrs). Cells were harvested and analyzed for PD-L1 expression by flow cytometry.

FIGS. 8A and 8B show that Platelet-derived TREML1 is enriched in the tumor. (A) TREML1 expression in human tissue was examined by immunostaining in formalin-fixed paraffin-embedded tissue, and the intensity of TREML1 expression in most human tumors was significantly higher than in normal tissue. The percentage of TREML-1-bound macrophages is quantitated, and the fold of change was calculated by dividing the percentage of TREML1/CD68 double positive cells in tumor tissues by that in normal tissue adjacent to the tumor (NAT). Since zero percentage of TREML1/CD68 double positive cells was found in some normal tissue, including Nose, Breast, Uterus, Prostate, Ovary, Kidney, Skin, Esophagus, and Stomach (marked with asterisk in FIG. 8A), the percentage of one, instead of zero, was used for calculating the fold of change. (B) The TREML1 (Brown color) can be found colocalized with macrophages (Green color) and distributed around the tumor stromal region. The representative picture of human stomach cancer tissue shows the site of TREML1 and macrophage colocalization (marked by arrows) and the representative picture of colon cancer shows TREML1 in a tumor (marked by an asterisk).

FIG. 9 shows that TREML1 is highly enriched in the tumor microenvironment. In vivo monitoring of TREML1 in MC38 tumor. IVIS image of MC38 tumor-bearing mice after receiving VivoTag680XL-conjugated isotype control IgG (MOPC21) or anti-TREML1 antibody (26A6). The IVIS image from anti-TREML1 antibody-treated mice shows a positive bioluminescent signal (white arrow) in the MC38 tumor area.

FIG. 10 shows the anti-cancer effects of anti-TREML1 antibodies (26A6 and 23F10) in the MC38 colon cancer model. MC38 tumor-bearing mice were treated intraperitoneally with 10 mg/kg of anti-TREML1 or isotype control IgG (MOPC21) antibodies twice per week. Mice were monitored and scored for the formation of palpable tumors twice weekly.

### DETAILED DESCRIPTION

The immune system can be divided into two categories or subsystems, the innate immune system, and the adaptive immune system. Innate immunity refers to the nonspecific defense mechanism that responds immediately or within hours of an antigen's presence in a subject. These defense mechanisms include physical barriers such as the skin, chemicals in the blood, and immune cells that attack foreign cells in the body. The innate immune response is activated by the chemical properties of an antigen. In contrast, adaptive immunity refers to antigen-specific immune responses and requires the recognition of specific “non-self” antigens during the antigen presentation process. The adaptive immune response provides a tailored response to each stimulus by learning to recognize molecules it has previously encountered. Adaptive immunity also includes memory cells that maintain tailored responses to the specific antigen, so that response to future attacks by the same antigen is more efficient.

Triggering receptors expressed by myeloid cells (TREMs) belong to a family of receptors that include activating and inhibitory isoforms encoded by a gene cluster linked to the MHC. TREM1 activates myeloid cells by signaling through the adaptor protein DAP12. TREM1 triggers phagocyte secretion of pro-inflammatory chemokines and cytokines, amplifying the inflammation that is induced by bacteria and fungi.

The present disclosure is based on the unexpected finding that TREML1 ECD (soluble TREML1, soluble TLT1) binds to the I-domain of CD11b and induces immune suppression and that anti-CD 11b antibodies can reverse TREML1 ECD-induced immune suppression. The antibodies described herein inhibit or reduce TREML1 ECD binding to CD11b+ immune cells and reverse TREML1 ECD induced immune suppression. The present disclosure also describes the use of the anti-TREML1 antibodies described herein to treat various immune suppressive disorders including cancer, sepsis, infections, autoimmune diseases, chronic diseases, immune exhaustion, or immunosenescence in aging.

CD11b is constitutively expressed on the surface of leukocytes including macrophages, monocytes, neutrophils, dendritic cells, natural killer cells, and granulocytes, which are the main players of the innate immune response cell network, and CD11 b is also conditionally expressed on a subset of T cells (J. Immunol. 2001 Jan. 15; 166(2):900-7) and a subset of B cells (Proc. Natl. Acad. Sci. U.S.A. 2008 Apr. 1; 105(13):5195-200). Accordingly, the binding of TREML1 to the I-domain of CD11 b is associated with an innate immune response. In contrast, the binding of TREML1 to CD3+ and CD8+ T cells is associated with adaptive immune response.

### Definitions

The terms “a,” “an,” “the” and similar referents used in the context of describing the claimed subject matter (especially in the context of the following claims) are to be construed to cover both the singular and the plural, unless otherwise indicated herein or clearly contradicted by context.

Amino acid residues are abbreviated as follows: alanine (Ala, A), asparagine (Asn, N), aspartic acid (Asp, D), arginine (Arg, R), cysteine (Cys, C), glutamic acid (Glu, E), glutamine (Gln, Q), glycine (Gly, G), histidine (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V).

As will be understood by one of ordinary skill in the art, each embodiment disclosed herein can comprise, consist essentially of, or consist of its particular stated element, step, ingredient, or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment. In embodiments, those elements or steps that do not affect an embodiment are those elements or steps that do not alter the embodiment's ability in a statistically significant manner to perform a function in vitro or in vivo, such as killing cancer cells in vitro or in vivo.

The term “affinity” refers to the strength of the total noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of molecule X for its partner Y can generally be represented by the dissociation constant (Kd). Affinity can be measured by common methods known in the art, including those described herein.

As used herein, the term “antibody” is used in the broadest sense and specifically covers monoclonal antibodies (including full-length monoclonal antibodies), polyclonal antibodies, multi-specific antibodies (e.g., bispecific antibodies), monovalent antibodies, multivalent antibodies, and antibody fragments so long as they exhibit the desired biological activity (e.g., Fab and/or single-armed antibodies).

The term “antibody fragment” refers to an antibody fragment that comprises a portion of an intact antibody. An antibody fragment can retain its binding to the antigen that the intact antibody binds. Examples of such antibody fragments include Fv, Fab, Fab′, Fab′-SH, F (ab′) 2, diabodies, linear antibodies, single chain antibodies (scFv), and multi-specific antibodies formed from antibody fragments.

The term “antigen-binding fragment” or “antigen-binding portion” of an antibody, refers to one or more portions of an antibody that retain the ability to specifically bind to an antigen. It has been shown that the antigen-binding function of an antibody can be performed by fragments of a full-length antibody. Examples of binding fragments encompassed within the term “antigen-binding fragment” of an antibody include (i) a Fab fragment, a monovalent fragment consisting of the VL, VH, CL, and CH1 domains; (ii) a F(ab′)2 fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody; (v) a dAb fragment which consists of a VH domain; and (vi) an isolated complementarity determining region (CDR). These antibody fragments are obtained using conventional procedures, such as proteolytic fragmentation procedures, as described in J. Goding, Monoclonal Antibodies: Principles and Practice, pp 98-118 (N.Y. Academic Press 1983). The fragments are screened for utility in the same manner as the intact antibodies.

As used herein, the term “complementarity determining regions” (CDRs) refers to the regions within antibodies where these proteins complement an antigen's shape. The acronym CDR is used herein to mean “complementarity determining region.” A single antibody has two antigen receptors, so it has twelve CDRs. There are three CDR loops per variable region in antibodies.

A “variable region” of an antibody refers to the variable region of the antibody light chain or the variable region of the antibody heavy chain, either alone or in combination. The variable regions of the heavy and light chains each consist of four framework regions (FR) connected by three CDRs also known as hypervariable regions. The CDRs in each chain are held together in close proximity by the FRs and, with the CDRs from the other chain, contribute to the formation of the antigen-binding site of antibodies. Exemplary conventions that can be used to identify the boundaries of CDRs include, e.g., the Kabat definition and the Chothia definition. The Kabat definition is based on sequence variability (see Kabat et al., 1992, Sequences of Proteins of Immunological Interest, 5th ed., Public Health Service, NIH, Washington D.C.), the Chothia definition is based on the location of the structural loop regions (Chothia et al., 1989, Nature 342: 877-883). Other approaches to CDR identification include the “IMGT definition” (Lefranc, M. -P. et al., 1999, Nucleic Acids Res. 27: 209-212) and the “AbM definition,” which is a compromise between Kabat and Chothia and is derived using Oxford Molecular's AbM antibody modeling software, or the “contact definition” of CDRs based on observed antigen contacts, set forth in MacCallum et al., 1996, J. Mol. Biol. 262: 732-745. As used herein, a CDR may refer to CDRs defined by the Kabat numbering system.

The term “humanized antibody” or a “humanized antibody fragment” is a specific type of chimeric antibody that includes an immunoglobulin amino acid sequence variant, or fragment thereof, which is capable of binding to a predetermined antigen and which, comprises one or more frameworks (FRs) having substantially the amino acid sequence of a human immunoglobulin and one or more complementarity determining regions (CDRs) having substantially the amino acid sequence of a non-human immunoglobulin. This non-human amino acid sequence often referred to as an “import” sequence is typically taken from an “import” antibody domain, particularly a variable region. In general, a humanized antibody includes at least the CDRs or hypervariable regions (HVLs) of a non-human antibody, inserted between the FRs of a human heavy or light chain variable region.

The term “human antibody” refers to an antibody that possesses an amino acid sequence that corresponds to that of an antibody produced by a human or a human cell or derived from a non-human source that utilizes human antibody repertoires or other human antibody-encoding sequences. A human antibody specifically excludes a humanized antibody comprising non-human antigen-binding residues.

The term “chimeric antibody” refers to an antibody that contains one or more regions from one antibody and one or more regions from one or more other antibodies.

As used herein, the term “heavy chain” includes a full-length heavy chain and fragments thereof having sufficient variable region sequence to confer specificity to an epitope. A full-length heavy chain includes a variable region, VH or VH, and three constant region domains, CH1, CH2, and CH3. The VH domain is at the amino-terminus of the polypeptide, and the CH3 domain is at the carboxyl-terminus.

The term “light chain” includes a full-length light chain and fragments thereof having sufficient variable region sequence to confer specificity to an epitope. A full-length light chain includes a variable region, VL or VL, and a constant domain, C L. Like the heavy chain, the variable region of the light chain is at the amino-terminus of the polypeptide.

The term “CD11b” refers to integrin alpha M (ITGAM, CR3A), which is one subunit of the heterodimeric integrin αMβ2. The second subunit of integrin αMβ2 is the common integrin β2 subunit known as CD18. Integrin αMβ2 is also called macrophage-1 antigen (Mac-1) or complement receptor 3 (CR3) which is constitutively expressed on the surface of leukocytes including monocytes, neutrophils, granulocytes, macrophages, dendritic cells, and natural killer cells, and conditionally expressed on a subset of T cells (J. Immunol. 2001 Jan. 15; 166(2):900-7) and a subset of B cells (Proc. Natl. Acad. Sci. U.S.A. 2008 Apr. 1; 105(13):5195-200).

The term “PD-L1” refers to programmed death-ligand 1 (PD-L1), cluster of differentiation 274 (CD274), or B7 homolog 1 (B7-H1). PD-L1 is a transmembrane protein that plays a major role in suppressing the immune system.

The term “PD-1” refers to programmed death protein 1, also known as CD279. It is a protein on the surface of T and B cells that down-regulate the immune system. PD-1 plays an important role in inhibiting immune responses and promoting self-tolerance by modulating the activity of T cells. PD-1 and its ligand PD-L1 inhibit T cell activation, proliferation, survival, and cytotoxic secretion within cancer cells. It has been reported that PD-1 and PD-L1 are involved in suppressing the immune system in autoimmune diseases, cancer, rheumatoid arthritis, neurodegenerative diseases, sepsis, and other infectious diseases such as Mycobacterium tuberculosis, cytomegalovirus, and hepatitis.

The term “monocyte,” also called a mononuclear white cell, belongs to a type of white blood cell involved in first-line defensive mechanism and is recognized as able to differentiate into a dendritic cell or macrophage precursor. Monocytes normally move in the blood system. In response to external stimulating signals, monocytes secrete many immuno-regulatory cytokines, move to the site of infection in the tissues, and differentiate into macrophages.

The term “modulating” includes “increasing”, “inducing”, “promoting,” or “stimulating, “as well as “decreasing”, “reducing”, or “inhibiting” in a statistically significant or physiologically significant amount as compared to a control.

The terms “inhibiting” and “reversing” are used interchangeably in the context of immune suppression to mean alleviating or reducing the suppression of the immune response.

The term “pharmaceutically acceptable carrier” refers to an ingredient in a pharmaceutical formulation, other than an active ingredient, which is nontoxic to a subject. A pharmaceutically acceptable carrier includes a buffer, excipient, stabilizer, or preservative.

The term “effective amount” refers to an amount sufficient to effect beneficial or desired clinical results. An effective amount can be administered in one or more administrations.

The term “therapeutic” refers to a treatment and/or prophylaxis. A therapeutic effect is obtained by suppression, remission, or eradication of a disease state or alleviating the symptoms of a disease state.

The term “therapeutically effective amount” refers to the amount of a composition or an agent that will elicit the biological or medical response of a tissue, system, or subject that is being sought by the researcher, veterinarian, medical doctor, or another clinician. The term “therapeutically effective amount” includes the amount of a compound that, when administered, is sufficient to prevent the development of, or alleviate to some extent, one or more of the signs or symptoms of the disease or condition being treated. The therapeutically effective amount will vary depending on the agent, the disease and its severity, and the age, weight, etc., of the subject to be treated. A therapeutically effective amount is an amount that is sufficient to diagnose, palliate, ameliorate, stabilize, reverse, inhibit, slow or delay the progression of a disease state or a cellular process.

The terms “treatment”, “treating”, or “treat” and the like generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. Treatment includes any treatment of a disease in a mammal, particularly a human, and includes: (a) inhibiting one or more disease symptoms, i.e., arresting its development, from occurring in a subject; or (b) relieving, ameliorating, or alleviating one or more disease symptoms, i.e., causing regression of the disease or symptom, in a subject.

The term “prevent” or “prevention” refers to a preventative or prophylactic measure that prevents the onset, recurrence, or spread of a disease state or condition or one or more symptoms of the disease state or condition from occurring in a subject. The term includes the administration of a composition or an agent described herein before the onset of symptoms to a subject at risk of developing a disease state or condition. The term includes the inhibition or reduction of one or more symptoms associated with a disease state or condition. The term “prevention” can be used interchangeably with the term “prophylactic treatment”.

The term “immune checkpoint blockade (ICB) therapy” refers to cancer immunotherapy using immune checkpoint inhibitors such as anti-PD-L1, anti-CTLA-4, and anti-PD-1 antibodies. Anti-PD-1, anti-CTLA-4, and anti-PD-L1 monoclonal antibodies have been reported to be reported to provide durable responses in patients with different cancers.

The term “responsive to therapies that can be administered as a form of cancer treatment” or “responsive to cancer therapies” refers to a subject diagnosed with cancer or the growth of a malignant tumor and who will respond or benefit from one or more therapies that can be administered to treat the cancer. The subject will not be resistant to the one or more therapies and will respond or benefit from the therapy, such as inhibition of the growth of the cancer cells or the tumor or the reduction of the size of the cancer cells or tumor. Examples of cancer therapies include immunotherapy including ICB therapy.

The term “all therapies” or “therapies” can be used interchangeably to include one or more cancer therapies and/or various cancer therapies that can be administered as a form of cancer treatment.

The term “subject” includes a human or non-human animal. Non-human animals include mammals, such as mice, rats, dogs, pigs, monkeys, and apes, and non-mammalian animals, such as birds, reptiles, fish, and amphibians. A subject in need of treatment or in need thereof includes a subject having a disease or condition that needs to be treated. A subject in need thereof also includes a subject that needs treatment or prevention of a disease or condition. In embodiments, the disease or condition is an immune suppressive disease including cancer.

The term “sequence identity” refers to a relationship between two or more sequences of polypeptides or polynucleotides, as determined by comparing their sequences. The term “sequence identity” also refers to the degree of sequence relatedness between polypeptides or polypeptides, as determined by the matches between strings of their sequences. Sequence Identity can be easily calculated by known bioinformatical methods. As an example, the “percent identity” of two polynucleotides or two polypeptide sequences is determined by comparing the sequences using the GAP computer program (a part of the GCG Wisconsin Package, version 10.3 (Accelrys, San Diego, Calif.)) using its default parameters.

Recitation of ranges of values herein is merely intended to serve as a shorthand method of referring individually to each separate value falling within the range. Unless otherwise indicated herein, each individual value is incorporated into the specification as if it were individually recited herein. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.5, 2.7, 3, 4, 5, 5.1, 5.3, 5.8, and 6. This applies regardless of the breadth of the range. Moreover, any ranges cited herein are inclusive of the upper and lower limit of the ranges.

When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; 15% of the stated value; 10% of the stated value; 5% of the stated value; 4% of the stated value; 3% of the stated value; 2% of the stated value; 1% of the stated value; or ±any percentage between 1% and 20% of the stated value.

Agents that Bind TREML1 and Compositions Thereof

The present disclosure describes agents that bind TREML1 and reverse immune suppression induced by TREML1 binding to CD11b. Agents that bind TREML1 include any compounds or molecules that bind TREML1 ECD and can reverse the immune suppression induced by TREML1 binding to CD11b. In embodiments, the agent described herein includes an antibody that binds TREML1 ECD.

The antibodies described herein include monoclonal antibodies, polyclonal antibodies, chimeric antibodies, bispecific antibodies, humanized antibodies, and antigen-binding fragments thereof. These antibodies bind TREML1 and reverse immune suppression induced by TREML1 binding to CD11b. In embodiments, these antibodies bind TREML1 ECD.

The antibodies described herein can be obtained by any known method including phage display technology, recombinant technology, computational technology, hybridoma technology, or immunizing an animal. As an example, the antibodies described herein are obtained by immunizing the mice with human TREML1 antigen and then screening and isolating hybridomas carrying specific antibody genes that can recognize TREML1 ECD.

Exemplary antibodies that bind TREML1 are provided in Tables 1-3 below. As shown by the consensus or conserved sequence of Table 1, the present disclosure describes antibodies that bind TREML1 including a VH region comprising amino acids SEQ ID NO: 20 (CDR-H1), SEQ ID NO: 29 (CDR-H2), and SEQ ID NO: 30 (CDR-H3), and a VL region comprising amino acids SEQ ID NO: 31 (CDR-L1), SEQ ID NO: 24 (CDR-L2), and SEQ ID NO: 25 (CDR-L3). As shown by the consensus or conserved sequence of Table 2, the present disclosure describes antibodies that bind TREML1 including a VH region comprising amino acids SEQ ID NO: 49 (CDR-H1), SEQ ID NO: 50 (CDR-H2), and SEQ ID NO: 51 (CDR-H3), and a VL region comprising SEQ ID NO: 52 (CDR-L1), SEQ ID NO: 53 (CDR-L2), and SEQ ID NO: 54 (CDR-L3). In embodiments, amino acid “X” in the amino acid sequence of the CDRs can be any amino acid.

Amino acids differ from each other with respect to their side chains (or R groups). For example, their side chains can differ in structure, electrical charge, and polarity. There are three types of polar amino acids neutral amino acids, positively charged amino acids, and negatively charged amino acids. Polar amino acids with uncharged side chains include Serine (S), Threonine (T), Cysteine (C), Proline (P), Asparagine (N), and Glutamine (Q). Polar amino acids with positively charged side chains include Lysine (K), Arginine (R), and Histidine (H), and polar amino acids with negatively charged side chains include Aspartic Acid (D) and Glutamic Acid (E). Nonpolar amino acids with aliphatic or hydrophobic side chains include Alanine (A), Isoleucine (I), Leucine (L), Methionine (M), Glycine (G), and Valine (V), and nonpolar amino acids with aromatic side chains include Phenylalanine (F), Tryptophan (W), and Tyrosine (Y).

In embodiments, based on the sequences shown in Tables 1 and 2, amino acid “X” can be a specific amino acid, as shown in the following examples. In SEQ ID NO: 29, the first amino acid X can include a polar amino acid with an uncharged or a positively charged side chain, such as S or R, and the second amino acid X can include a nonpolar amino acid with an aliphatic side chain, such as a V or I. In SEQ ID NO: 30, the first amino acid X can include a polar amino acid with an uncharged or negatively charged side chain, such as D or N, and the second amino acid X can include a nonpolar amino acid with an aliphatic side chain, such as I or M. In SEQ ID NO: 31, amino acid X can include a polar amino acid with an uncharged side chain, such as N or S. In SEQ ID NO: 49, the first amino acid X can include a polar amino acid with a negatively charged or uncharged side chain, such as D, E, or N, and the second amino acid X can include a nonpolar amino acid with an aliphatic side chain, such as I or M. In SEQ ID NO: 50, the first amino acid X can include a nonpolar amino acid with an aliphatic or aromatic side chain, such as I, M, or F; the second amino acid X can include a polar amino acid with uncharged side chain, such as T or N; the third amino acid X can include a polar amino acid with a negatively charged side chain, such as D, or a nonpolar amino acid with aliphatic side chain, such as G; the fourth amino acid X can include a nonpolar amino acid with an aliphatic side chain, such as G, or a polar amino acid with a positively charged side chain, such as H; the fifth amino acid X can include a polar amino acid with an uncharged side chain, such as P or S, or a nonpolar amino acid with an aliphatic side chain, such as A; the sixth amino acid X can include a nonpolar amino acid with an aromatic side chain, such as amino acid Y or F; the seventh amino acid X can include a polar amino acid with an uncharged side chain, such as S or N; the eighth amino acid X can include a polar amino acid with negatively charge side chain, such as D or E; the ninth amino acid X can include a polar amino acid with a positively charged or uncharged side chain, such as K or T; the tenth amino acid X can include a nonpolar amino acid with an aliphatic or aromatic side chain, such as I, A, or F; the eleventh amino acid X can include a polar amino acid with a positively charged or uncharged side chain, such as K, R, or T; and the twelfth amino acid X can include a polar amino acid with a negatively charged side chain, such as D, or a nonpolar amino acid with an aliphatic side chain, such as G. In SEQ ID NO: 51, amino acid X can include a nonpolar amino acid with an aromatic side chain, such as F or Y. In SEQ ID NO: 52, the first amino acid X can include a polar amino acid with a positively charged side chain, such as R or K; the second amino acid X can include a polar amino acid with an uncharged side chain, such as S or T; the Clean Substitute Specification Attorney Docket No. A263-1005U third amino acid X can include a nonpolar amino acid with an aliphatic side chain, such as L, V. or 1; and the fourth amino acid X can include a nonpolar amino acid with an aliphatic side chain, such as L or V. In SEQ ID NO: 53, amino acid X can include a polar amino acid with a positively charged or uncharged side chain, such as K or Q. In SEQ ID NO: 54, the first amino acid X can include a polar amino acid with an uncharged side chain, such as T or S; the second amino acid X can include a polar amino acid with a positively charged side chain, such as H, or a nonpolar amino acid with an aromatic side chain, such as Y; and the third amino acid X can include a nonpolar amino acid with an aliphatic side chain, such as I or V. The “X” amino acids in each sequence described above are numbered sequentially with the first X amino being closest to the amino terminus of the peptide.

TABLE 1
CDRs of Exemplary Antibodies
Table 1(A): CDRs of Heavy Chain Variable Region

| Clone ID | CDR-H1 | CDR-H2 | CDR-H3 |
|---|---|---|---|
| 17E6 | DYGMA | FISNLAYSVYYADTVTG | EDYGDNGAIDY |
| | (SEQ ID | (SEQ ID NO: 21) | (SEQ ID NO: |
| | NO: 20) | | 22) |
| 25C6 | DYGMA | FISNLAYSVYYADTVTG | EDYGDNGAIDY |
| | (SEQ ID | (SEQ ID NO: 21) | (SEQ ID NO: |
| | NO: 20) | | 22) |
| 26A6 | DYGMA | FISNLAYRIYYADTVTG | EDYGNNGAMDY |
| | (SEQ ID | (SEQ ID NO: 27) | (SEQ ID NO: |
| | NO: 20) | | 28) |
| Conserved | DYGMA | FISNLAYXXYYADTVTG | EDYGXNGAXDY |
| sequence | (SEQ ID | (SEQ ID NO: 29) | (SEQ ID NO: |
| | NO: 20) | | 30) |

Note:
“X” can be any amino acid

TABLE 1(B)
CDRs of Light Chain Variable Region

| Clone ID | CDR-L1 | CDR-L2 | CDR-L3 |
|---|---|---|---|
| 17E6 | RSSQNIVHSNGNTYLE | KVSNRFS | FQGSHVPPT |
| | (SEQ ID NO: 23) | (SEQ ID | (SEQ ID |
| | | NO: 24) | NO: 25) |
| 25C6 | RSSQSIVHSNGNTYLE | KVSNRFS | FQGSHVPPT |
| | (SEQ ID NO: 26) | (SEQ ID | (SEQ ID |
| | | NO: 24) | NO: 25) |
| 26A6 | RSSQSIVHSNGNTYLE | KVSNRFS | FQGSHVPPT |
| | (SEQ ID NO: 26) | (SEQ ID | (SEQ ID |
| | | NO: 24) | NO: 25) |
| Conserved | RSSQXIVHSNGNTYLE | KVSNRFS | FQGSHVPPT |
| sequence | (SEQ ID NO: 31) | (SEQ ID | (SEQ ID |
| | | NO: 24) | NO: 25) |

Note:
“X” can be any amino acid

TABLE 2
CDRs of Exemplary Antibodies
Table 2(A): CDRs of Heavy Chain Variable Region

| Clone ID | CDR-H1 | CDR-H2 | CDR-H3 |
|---|---|---|---|
| 18D8 | DYVIH | YMNPYTDGPKYSDKIKD | DFNYYVGAMDF |
| | (SEQ ID | (SEQ ID NO: 33) | (SEQ ID |
| | NO: 32) | | NO: 34) |
| 23F10 | DYVIH | YINPYTGGPKYSETARG | DFNYYVGAMDF |
| | (SEQ ID | (SEQ ID NO: 37) | (SEQ ID |
| | NO: 32) | | NO: 34) |
| 27D3 | EYVIH | YFNPYTGGSKFNEKFKD | DFNYYVGAMDY |
| | (SEQ ID | (SEQ ID NO: 40) | (SEQ ID |
| | NO: 39) | | NO: 55) |
| 28A9 | NYVMH | YFNPYNGHAKYSEKFTG | DFNYYVGAMDY |
| | (SEQ ID | (SEQ ID NO: 44) | (SEQ ID |
| | NO: 43) | | NO: 55) |
| 29F4 | DYVIH | YINPYTGGPKYSETAKG | DFNYYVGAMDF |
| | (SEQ ID | (SEQ ID NO: 47) | (SEQ ID |
| | NO: 32) | | NO: 34) |
| Conserved | XYVXH | YXNPYXXXXKXXXXXXX | DFNYYVGAMDX |
| sequence | (SEQ ID | (SEQ ID NO: 50) | (SEQ ID |
| | NO: 49) | | NO: 51) |

Note:
“X” can be any amino acid

TABLE 2(B)
CDRs of Light Chain Variable Region

| Clone ID | CDR-L1 | CDR-L2 | CDR-L3 |
|---|---|---|---|
| 18D8 | RSSQSLLHSNGNTYLH | KVSNRFS | SQSTHIWT |
| | (SEQ ID NO: 35) | (SEQ ID | (SEQ ID |
| | | NO: 24) | NO: 36) |
| 23F10 | RSTQSLVHSNGNTYVH | KVSNRFS | SQSTYVWT |
| | (SEQ ID NO: 38) | (SEQ ID | (SEQ ID |
| | | NO: 24) | NO: 56) |
| 27D3 | RSSQSLVHSNGNTYLH | KVSNRFS | SQSSHIWT |
| | (SEQ ID NO: 41) | (SEQ ID | (SEQ ID |
| | | NO: 24) | NO: 42) |
| 28A9 | RSSQSLIHSNGNTYLH | QVSKRFS | SQSTHIWT |
| | (SEQ ID NO: 45) | (SEQ ID | (SEQ ID |
| | | NO: 46) | NO: 36) |
| 29F4 | KSTQSLVHSNGNTYVH | KVSNRFS | SQSTYVWT |
| | (SEQ ID NO: 48) | (SEQ ID | (SEQ ID |
| | | NO: 24) | NO: 56) |
| Conserved | XSXQSLXHSNGNTYXH | XVSNRFS | SQSXXXWT |
| sequence | (SEQ ID NO: 52) | (SEQ ID | (SEQ ID |
| | | NO: 53) | NO: 54) |

Note:
“X” can be any amino acid

In embodiments, anti-TREML1 antibodies include various clones including V_{H} and V_{L}regions comprising amino acid sequences shown in Table 3. Exemplary antibodies described herein include clone 17E6 comprising amino acids SEQ ID NO: 4 (V_{H}) and SEQ ID NO: 5 (V_{L}); Clean Substitute Specification Attorney Docket No. A263-1005U clone 2506 comprising SEQ ID NO: 6 (V_{H}) and SEQ ID NO: 7 (V_{L}); clone 26A6 comprising SEQ ID NO: 8 (V_{H}) and SEQ ID NO: 9 (V_{L}); clone 1808 comprising SEQ ID NO: 10 (V_{H}) and SEQ ID NO: 11 (V_{L}); clone 23F10 comprising SEQ ID NO: 12 (V_{H}) and SEQ ID NO: 13 (V_{L}); clone 2703 comprising SEQ ID NO: 14 (V_{H}) and SEQ ID NO: 15 (V_{L}) clone 28A9 comprising SEQ ID NO: 16 (V_{H}) and SEQ ID NO: 17 (V_{L}) and clone 29F4 comprising SEQ ID NO: 18 (V_{H}) and SEQ ID NO: 19 (V_{L}).

### Table 3: Exemplary Anti-TREML E Antibodies

The CDR sequences for each antibody are underlined.

TABLE 3(A)
Anti-TREML1 Antibody Clone 17E6

| Sequence | | |
|---|---|---|
| ID number: | Description | Sequence |
| SEQ ID | Heavy chain | EVKLVESGGGLVQPGGSRKLSCAAS |
| NO: 4 | variable | GFTFSDYGMAWVRQAPGKGPEWIAF |
| | | ISNLAYSVYYADTVTGRFTISRENA |
| | | KNTLYLEMSSLRSEDTAMYYCARED |
| | | YGDNGAIDYWGQGTSVTVSS |
| SEQ ID | Light chain | DVLMTQTPLSLPVSLGDQASISCRS |
| NO: 5 | variable | SQNIVHSNGNTYLEWYLQKPGQSPQ |
| | | LLIYKVSNRFSGVPDRFSGSGSGTD |
| | | FTLKISRVEAEDLGVYYCFQGSHVP |
| | | PTFGGGTKLEIK |

TABLE 3(B)
Anti-TREML1 Antibody Clone 25C6

| Sequence | | |
|---|---|---|
| ID number: | Description | Sequence |
| SEQ ID | Heavy chain | EVKLVESGGGLVQPGGSRKLSCAAS |
| NO: 6 | variable | GFTFSDYGMAWVRQAPGKGPEWIAF |
| | | ISNLAYSVYYADTVTGRFTISRENA |
| | | KNTLYLEMSSLRSEDTAMYYCARED |
| | | YGDNGAIDYWGQGTSVTVSS |
| SEQ ID | Light chain | DVLMTQTPLSLPVSLGDQASISCRS |
| NO: 7 | variable | SQSIVHSNGNTYLEWYLQKPGQSPK |
| | | LLIYKVSNRFSGVPDRFSGSGSGTD |
| | | FTLKISRVEAEDLGVYYCFQGSHVP |
| | | PTFGGGTKLEIK |

TABLE 3(C)
Anti-TREML1 Antibody Clone 26A6

| Sequence | | |
|---|---|---|
| ID number: | Description | Sequence |
| SEQ ID | Heavy chain | EVKLVESGGGLVQPGGSRKLSCAAS |
| NO: 8 | variable | GFTFSDYGMAWVRQAPGKGPEWIAF |
| | | ISNLAYRIYYADTVTGRFTISRENA |
| | | KNTLYLEMSSLRSEDSAIYYCVRED |
| | | YGNNGAMDYWGQGTSVTVSS |
| SEQ ID | Light chain | DVLMTQTPLSLPVSLGDQASISCRS |
| NO: 9 | variable | SQSIVHSNGNTYLEWYLQKPGQSPK |
| | | LLIYKVSNRFSGVPDRFSGSGSGTD |
| | | FTLKISRVEAEDLGVYYCFQGSHVP |
| | | PTFGGGTKLEIK |

TABLE 3(D)
Anti-TREML1 Antibody Clone 18D8

| Sequence | | |
|---|---|---|
| ID number: | Description | Sequence |
| SEQ ID | Heavy chain | EVQVQQSGPELVNPGASVRLSCKAS |
| NO: 10 | variable | GYIFTDYVIHWVRQKPGQGLEWIGY |
| | | MNPYTDGPKYSDKIKDKAILTSDRS |
| | | SNTVYMELRSLTSEDSAVYFCARDF |
| | | NYYVGAMDFWGQGTSVTVSS |
| SEQ ID | Light chain | DVVLTQTPLSLPVSLGDQASVSCRS |
| NO: 11 | variable | SQSLLHSNGNTYLHWYLQKPGQSPK |
| | | LLIYKVSNRFSGVPDRFTGSGSGTD |
| | | FTLKISRVEAEDLGVYFCSQSTHIW |
| | | TFGGGTKLEIK |

TABLE 3(E)
Anti-TREML1 Antibody Clone 23F10

| Sequence | | |
|---|---|---|
| ID number: | Description | Sequence |
| SEQ ID | Heavy chain | EVQLQQSGPELVRPGASVKVSCKAS |
| NO: 12 | variable | GYIFTDYVIHWVKQKPGQGLEWFGY |
| | | INPYTGGPKYSETARGKATLTSDSS |
| | | SNTAYMVLTSLTSDDSAVYYCTRDF |
| | | NYYVGAMDFWGQGTSVTVSS |
| SEQ ID | Light chain | AAVLTQTPLSLPVSLGDQASISCRS |
| NO: 13 | variable | TQSLVHSNGNTYVHWYLQKAGQSPK |
| | | LLIYKVSNRFSGVPDRFSGSGSGTD |
| | | FTLKISRVEAEDLGVYFCSQSTYVW |
| | | TFGGGTKLEIE |

TABLE 3(F)
Anti-TREML1 Antibody Clone 27D3

| Sequence | | |
|---|---|---|
| ID number: | Description | Sequence |
| SEQ ID | Heavy chain | DVQLQQSGPELVKPGASVKMSCKAS |
| NO: 14 | variable | GYTFTEYVIHWVKQRPGQGLEWFGY |
| | | FNPYTGGSKFNEKFKDKATLTSDRS |
| | | SNTVYMELYSLTSEDSAVYYCARDF |
| | | NYYVGAMDYWGQGTSVTVSS |
| SEQ ID | Light chain | DVALTQTPLSLPVSLGDQASISCRS |
| NO: 15 | variable | SQSLVHSNGNTYLHWYLQKSGQSPK |
| | | LLIYKVSNRFSGVPDRFSGSGSVTE |
| | | FTLKISRVEAEDLGVYFCSQSSHIW |
| | | TFGGGTRLEIK |

TABLE 3(G)
Anti-TREML1 Antibody Clone 28A9

| Sequence | | |
|---|---|---|
| ID number: | Description | Sequence |
| SEQ ID | Heavy chain | EVQLQQSGPELVKPGASVKMSCKAS |
| NO: 16 | variable | GYTFTNYVMHWVKQKPGQGLEWIGY |
| | | FNPYNGHAKYSEKFTGKATLTSDRS |
| | | SSTAYMELNSLTSEDSAVYYCTRDF |
| | | NYYVGAMDYWGQGTSVTVSS |
| SEQ ID | Light chain | DVVMTQSPLSLPVNLGDHASISCRS |
| NO: 17 | variable | SQSLIHSNGNTYLHWYLQKPGQSPK |
| | | LLVYQVSKRFSGVPDRFSGSGSGTD |
| | | FTLKISRVEAEDLGVYFCSQSTHIW |
| | | TFGGGTTLEIK |

TABLE 3(H)
Anti-TREML1 Antibody Clone 29F4

| Sequence | | |
|---|---|---|
| ID number: | Description | Sequence |
| SEQ ID | Heavy chain | EVQLQQSGPELVRPGASVKVSCKAS |
| NO: 18 | variable | GYIFTDYVIHWVKQKPGQGLEWFGY |
| | | INPYTGGPKYSETAKGKATLTSDSS |
| | | SNTAYMVLTSLTSEDSAVYYCTRDF |
| | | NYYVGAMDFWGQGTSVTVSS |
| SEQ ID | Light chain | GAVLTQTPLSLPVSLGDQASISCKS |
| NO: 19 | variable | TQSLVHSNGNTYVHWYLQKAGQSPK |
| | | LLIYKVSNRFSGVPDRFIGSGSGTD |
| | | FTLKISRVEAEDLGVYFCSQSTYVW |
| | | TFGGGTKLEIE |

The present disclosure also describes compositions comprising one or more agents that bind TREML1 and reverse immune suppression induced by TREML1 binding to 0011 b including the antibodies that bind TREML1 ECD described herein. The compositions can also include a carrier. In embodiments, the composition is a pharmaceutical composition. The pharmaceutical composition can include a pharmaceutically acceptable carrier.

The term “carrier” includes a diluent, adjuvant, or excipient that can be added to the composition and does not affect the active ingredient, particularly the antibodies described herein. Examples of adjuvants include complete and incomplete Freund's adjuvant, which are used with animals, particularly research animals. Pharmaceutically acceptable carriers include sterile liquids, such as water and oils, including those of petroleum, animal, vegetable, or from synthetic origins, such as peanut oil, soybean oil, mineral oil, sesame oil, and the like. Water is a preferred excipient when the pharmaceutical composition is administered intravenously.

Saline solutions and aqueous dextrose and glycerol solutions can also be employed as liquid excipients, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like.

The compositions or pharmaceutical compositions described herein can also contain minor amounts of wetting or emulsifying agents, or pH buffering agents. These compositions can take the form of solutions, suspensions, emulsions, tablets, pills, capsules, powders, sustained-release formulations, and the like. The pharmaceutical compositions can be prepared as formulations. Oral formulations can include standard carriers such as pharmaceutical grades of mannitol, lactose, starch, magnesium stearate, sodium saccharine, cellulose, magnesium carbonate, and the like. Such formulations contain a therapeutically effective amount of the antibody described herein, in purified form, together with a suitable amount of carrier to provide the form for proper administration to the subject. The formulation should suit the mode of administration.

The administration of the pharmaceutical compositions described herein can be carried out in any convenient manner, including by aerosol inhalation, injection, ingestion, transfusion, implantation, or transplantation. The compositions described herein also can be administered to a subject orally, topically, intranasally, enterally, rectally, buccally, vaginally, sublingually, subcutaneously, intradermally, intratumorally, intranodally, intramedullary, intramuscularly, intravenously, intracranially, intraperitoneally, or a combination thereof. The administration of the pharmaceutical composition can be in any manner that is effective to deliver a therapeutically and/or prophylactically effective amount of the conjugate described herein to the subject in need thereof.

The terms “an immunologically effective amount”, “an anti-tumor effective amount”, “a tumor-inhibiting effective amount”, “a therapeutic amount”, or “an effective amount” refers to the precise amount of the active ingredient, such as the antibody described herein, to be administered, which can be determined by a physician with consideration of individual differences in age, weight, tumor size, the extent of infection or metastasis, and condition of the subject.

The dosage for administering the pharmaceutical compositions described herein to a subject will vary with the precise nature of the condition being treated and the recipient of the treatment. The scaling of dosages for human administration can be performed according to art-accepted practices by a physician depending on various factors described herein. The optimal dosage and treatment regime for a particular subject can readily be determined by a physician, taking into account parameters such as physical, physiological, and psychological factors including target, body weight, stage of disease, and route of administration.

Exemplary doses of the pharmaceutical compositions including the agents such as the antibodies described herein can include 0.0001 mg/kg to 200 mg/kg body weight of a subject. The total daily dose can be 0.1 mg/kg to 50.0 mg/kg administered to a subject one to three times a day. Additional useful doses can often range from 0.1 μg/kg to 5 μg/kg, 5 μg/kg to 100 μg/kg, 50 μg/kg to 500 μg/kg, 450 μg/kg to 1 mg/kg, 1 mg/kg to 10 mg/kg, 5 mg/kg to 50 mg/kg, 25 mg/kg to 75 mg/kg, 50 mg/kg to 100 mg/kg, 75 mg/kg to 125 mg/kg, 100 mg/kg to 150 mg/kg, 125 mg/kg to 175 mg/kg, or 150 mg/kg to 200 mg/kg. The exemplary doses provided are based on the body weight of the subject.

### Uses of the Agents Described Herein

Embodiments relate to agents described herein that can reduce soluble TREML1 binding to CD11b+ immune cells and their uses for reversing TREML1-induced immune suppression. The anti-TREML1 antibodies provide a novel and potential therapy for diseases and conditions associated with immune suppression.

The agents described herein include anti-TREML1 antibodies that bind TREML1 on the HEK293/TREML1 cells and CD11b+ immune cells. As a result of anti-TREML1 antibodies binding innate immune cells and competition with TREML1 ECD for binding innate immune cells, anti-TREML1 antibodies can be used to treat cancer.

The present disclosure describes methods administering one or more agents including antibodies described herein and pharmaceutical compositions described herein to subjects in need thereof for treating or alleviating one or more symptoms of a disease or condition associated or characterized by immune suppression. In embodiments, reversing immune suppression comprises inhibiting an inflammatory response, such as a monocyte-mediated, macrophage-mediated, and/or neutrophil-mediated inflammatory response. In embodiments, the disease or condition includes growth of a malignant tumor, tumor angiogenesis, cancer, chronic infection, sepsis, immune exhaustion, or immunosenescence in aging.

Examples of cancer and malignant tumor include melanoma, lung cancer, squamous cell carcinomas of the lung, head and neck cancer, breast cancer, ovarian cancer, uterine cancer, prostate cancer, gastric carcinoma, cervical cancer, esophageal carcinoma, bladder cancer, kidney cancer, brain cancer, liver cancer, colon cancer, bone cancer, pancreatic cancer, skin cancer, cutaneous or intraocular malignant melanoma, ovarian cancer, rectal cancer, cancer of the anal region, stomach cancer, testicular cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, esophagus cancer, small intestine cancer, endocrine system cancer, thyroid gland cancer, parathyroid gland cancer, adrenal gland cancer, sarcoma of soft tissue, urethra cancer, penis cancer, chronic or acute leukemia, solid tumors of childhood, lymphocytic lymphoma, carcinoma of the renal pelvis, neoplasm of the central nervous system (CNS), primary CNS lymphoma, spinal axis tumor, brain stem glioma, pituitary adenoma, Kaposi's sarcoma, epidermoid cancer, squamous cell cancer, T cell lymphoma, and gastrointestinal tract cancer. Examples of chronic or acute leukemia include acute myeloid leukemia, chronic myeloid leukemia, acute lymphoblastic leukemia, and chronic lymphocytic leukemia. In embodiments, the cancer or malignant tumor is colon cancer.

The present disclosure also describes methods of using one or more agents including the antibodies described herein or the pharmaceutical compositions described herein to inhibit the binding of TREML1 to CD11b.

Moreover, the present disclosure describes methods of using one or more agents including the antibodies described herein or the pharmaceutical compositions described herein to reverse immune suppression in cells.

Further, the present disclosure describes methods of using one or more agents including the antibodies described herein or the pharmaceutical compositions described herein to inhibit the growth of cancer cells or a malignant tumor.

Additionally, the present disclosure describes methods of using one or more agents including the antibodies described herein or the pharmaceutical compositions described herein to inhibit PD-L1 expression on cells. The method includes administering the agents including the antibodies described herein or the pharmaceutical compositions described herein to cells induced by TREML1 binding CD 11b to express PD-L1.

Furthermore, the present disclosure describes methods of using one or more anti-CD11 b antibodies to block TREML1 and CD11 b interaction and reverse TREML1-induced immunosuppression. The I-domain of CD11 b is the receptor for TREML1. Anti-CD11 b antibodies can normalize the levels of HLA-DR expression and reduce the levels of PD-L1 expression associated with immunosuppression.

In embodiments, the methods described herein include administering one or more agents including the antibodies described herein, or the pharmaceutical compositions described herein to cells in vitro, ex vivo, or in vivo, for example in a subject. The cells include immune cells. In embodiments, the cells include one or more cells of the immune response system. Examples of such cells include macrophages, neutrophils, monocytes, dendritic cells, natural killer cells, granulocytes, a subset of T cells (J. Immunol. 2001 Jan. 15; 166(2):900-7), and a subset of B cells (Proc. Natl. Acad. Sci. U.S.A. 2008 Apr. 1; 105(13):5195-200).

In embodiments, the methods described herein reverse the immune suppression of the cells and/or treat a disease or condition associated with immune suppression.

In embodiments, the methods described herein include administering one or more agents including the antibodies described herein or the pharmaceutical compositions described herein to immune cells. The immune cells can be cells of the innate immune response system. Examples of cells of the innate immune response system include macrophages, monocytes, dendritic cells, natural killer cells, neutrophils, and granulocytes. In embodiments, the cells are in a subject. In embodiments, the subject is a mammal.

In embodiments, the methods described herein reverse the immune suppression of the cells and/or treat a disease or condition associated with immune suppression. The methods described herein inhibit inflammation.

The present disclosure also describes TREML1 as a useful marker for diagnosing cancer and identifying subjects for various forms of cancer therapies. Thus, the present disclosure also describes methods for diagnosing cancer or detecting the presence of a malignant tumor in a subject and/or for determining whether a subject would be responsive to one or more therapies that can be administered as a form of cancer treatment. Such methods include obtaining a biological sample from the subject, and detecting the presence of TREML1 in the sample, thereby detecting cancer or malignant tumor in the subject and/or identifying the subject for one or more cancer therapies. In embodiments, the methods described herein further include quantifying the expression of TREML1 in the sample. The methods can further include comparing the expression of TREML1 in the subject to the expression of TREML1 in a control. The control can be a healthy subject or a reference value associated with expression of TREML1 in a healthy subject or a normal control. When the expression of TREML1 in the subject is higher than control, it indicates that the subject has cancer or a malignant tumor and/or is a candidate for one or more therapies that can be administered as a form of cancer treatment.

In embodiments, detecting the presence of TREML1 can include performing an immunological assay, a histological assay, a cytological assay, an enzyme-linked immunosorbent assay (ELISA), a bead-based detection assay, a DNA or RNA expression assay, or an aptamer-based assay. In embodiments, the methods are performed using one or more agents including antibodies described herein or the pharmaceutical compositions described herein.

In embodiments, the biological sample described herein includes body fluids, cells, or tissues, and optionally wherein body fluids include blood, urine, saliva, bile, bone marrow aspirate, breast milk, cerebral spinal fluid (CSF), plasma, serum, stool, vaginal fluid, or synovial fluid; optionally wherein cells include blood cells, epithelial cells, fibroblasts, hepatocytes, immune cells (such as T cells, B cells, NK cells, monocytes, macrophages, dendritic cells and the like), stem cells, peripheral blood cells, or stem cells; and optionally, wherein the tissue is from a biopsy or a resected tumor.

Examples of cancer and malignant tumor are described herein. In embodiments, the cancer or malignant tumor includes cancer of the gastrointestinal tract such as colon cancer.

In embodiments, subjects diagnosed with cancer or malignant tumor or identified for cancer therapy can be treated with one or more cancer therapies such as chemotherapy, radiation therapy, immunotherapy, and stem cell or bone marrow transplant therapy. As an example, subjects diagnosed or identified with cancer are candidates for immunotherapy including ICB therapy, if immune checkpoint blockade or immune check point inhibitors can be used to treat the subjects.

In embodiments, the methods further include treating the subject with ICB therapy. ICB therapies include checkpoint inhibitors and monoclonal antibody-based therapies that block the interaction of inhibitory receptors (immune checkpoints) expressed on the surface of immune cells, with their ligands. As an example, expression of PD-1 and PD-L1 are increased in certain cancer patients who responded to PD-1 blockade. ICB therapies associated with blocking PD-L1 include administering atezolizumab (Tecentriq®), avelumab (Fotivda®), or durvalumab (Imfinzi®). Atezolizumab is an FDA approved immunotherapy and has been shown to be effective in treating non-small cell lung cancer (NSCLC), small cell lung cancer, hepatocellular carcinoma, triple-negative breast cancer, and urothelial cancer including bladder cancer. Avelumab is an FDA-approved drug for treating skin cancer such as Merkel cell carcinoma and urothelial cancer. Durvalumab is an FDA approved immunotherapy for treating NSCLC.

ICB therapies using PD-1 inhibitors include pembrolizumab (Keytruda®), nivolumab (Opdivo®), and cemiplimab (Libtayo®). Pembrolizumab was approved for medical use in the U.S. in 2014 and has been used to treat melanoma, lung cancer, head and neck cancer, Hodgkin lymphoma, stomach cancer, cervical cancer, and certain types of breast cancer. Nivolumab has been approved for medical use since 2014 and has been used to treat melanoma, lung cancer, malignant pleural mesothelioma, renal cell carcinoma, Hodgkin lymphoma, head and neck cancer, urothelial carcinoma, colon cancer, esophageal squamous cell carcinoma, liver cancer, gastric cancer, and esophageal or gastroesophageal junction cancer. Cemiplimab has been used for the treatment of squamous cell skin cancer. In 2018, cemiplimab was approved for the treatment of patients with metastatic cutaneous squamous cell carcinoma (CSCC) or locally advanced CSCC that are not candidates for curative surgery or curative radiation.

Another example of checkpoint inhibitors is CTLA-4 inhibitors. CTLA-4 is a checkpoint protein on T cells that acts as a type of “off switch” to help keep the immune system in check. CTLA-4 downregulates the immune system. ICB therapies using CTLA-4 inhibitors include ipilimumab (Yervoy®). Ipilimumab turns off the inhibitory mechanism of cytotoxic T lymphocytes (CTLs) and boosts the body's immune response against cancer cells. Ipilimumab has been approved since 2011 for the treatment of skin cancer. It is undergoing clinical trials for the treatment of other cancers including NSCLC, SCLC, bladder cancer, and metastatic hormone-refractory prostate cancer.

In embodiments, the subjects diagnosed with cancer or malignant tumor, and/or identified to be responsive for cancer treatment can be treated with one or more agents including the antibodies described herein or the pharmaceutical compositions described herein.

In embodiments, the subjects can be treated with all therapies that can be administered as a form of cancer treatment, including immunotherapy such as ICB therapy, targeted therapy, chemotherapy, radiation therapy, surgery, and other cancer therapies. In embodiments, the subjects can be treated with a combination of therapies described herein. In embodiments, the subjects can be treated with therapies that can be administered as a form of cancer treatment and one or more agents including the antibodies described herein or the pharmaceutical compositions described herein.

The present disclosure also describes kits for diagnosing cancer, detecting malignant tumor, and/or identifying subjects for therapies that can be administered as a form of cancer treatment. In embodiments, the kits contain one or more agents including the antibodies described herein for detecting the expression of TREML1 in a biological sample from a subject. The kits can also include a container or a device for collecting a biological sample from a subject and a container or device for mixing the agent and the biological sample for detecting cancer or growth of malignant tumor, and/or determining whether a subject is a candidate for various therapies that can be administered as a form of cancer treatment. Moreover, the kits can include a reference standard or control for comparison. Further, the kits can include instructions for using the one or more agents.

Additionally, the present disclosure describes kits for treating a subject diagnosed with cancer. In embodiments, the kits include one or more agents including one or more antibodies described herein or the pharmaceutical compositions described herein. In embodiments, the kits include all therapies that can be administered as a form of cancer treatment and one or more agents such as the one or more antibodies described herein or the pharmaceutical compositions described herein. The kits can include means for performing one or more cancer therapies including an immunotherapy such as ICB, which includes administering a checkpoint inhibitor or an antibody as a form of cancer treatment. The kits can also include a means for administering the one or more agents described herein and/or one or more cancer therapies to the subject, such as a device or an apparatus. Furthermore, the kits can include instructions for using the one or more agents described herein or one or more agents described herein in combination with one or more cancer therapies to treat the subject.

The following exemplary embodiments and examples illustrate exemplary methods provided herein. These exemplary embodiments and examples are not intended, nor are they to be construed, as limiting the scope of the disclosure. It will be clear that the methods can be practiced otherwise than as particularly described herein. Numerous modifications and variations are possible in view of the teachings herein and, therefore, are within the scope of the disclosure.

### EXEMPLARY EMBODIMENTS

### The Following are Exemplary Embodiments

1. An agent that inhibits the binding of triggering receptor expressed by myeloid cell (TREM)-like transcript-1 (TREML1) to CD11b, optionally, wherein TREML1 is in a soluble form.
2. The agent of embodiment 1, wherein the CD11 b is expressed on immune cells, and optionally, wherein the CD11 b is constitutively expressed on one or more leukocytes including macrophages, monocytes, neutrophils, dendritic cells, natural killer cells, and granulocytes, and optionally, wherein CD11 b is conditionally expressed on a subset of T cells and B cells.
3. The agent of embodiment 1 or 2, wherein the agent comprises an anti-TREML1 antibody or an antigen-binding fragment thereof.
4. The agent of any one of embodiments 1-3, wherein the agent comprises an anti-TREML antibody or antigen-binding fragment thereof comprising
   i) a heavy chain variable (VH) region, wherein the VH region comprises a heavy chain complementarity determining region 1 (CDR-H1) comprising DYGMA (SEQ ID NO: 20); a CDR-H2 comprising FISNLAYX₁X₂YYADTVTG (SEQ ID NO: 29); and a CDR-H3 comprising EDYGX₃NGAX₄DY (SEQ ID NO: 30); and
   a light chain variable (VL) region, wherein the VL region comprises a light chain complementarity region 1 (CDR-L1) comprising RSSQX₅IVHSNGNTYLE (SEQ ID NO: 31); a CDR-L2 comprising KVSNRFS (SEQ ID NO: 24); and a CDR-L3 comprising FQGSHVPPT (SEQ ID NO: 25); or
   ii) a heavy chain variable (VH) region, wherein the VH region comprises a CDR-H1 comprising X₆YVX₇H (SEQ ID NO: 49); a CDR-H2 comprising YX₈NPYX₉X₁₀X₁₁X₁₂KX₁₃X₁₄X₁₅X₁₆X₁₇X₁₈X₁₉ (SEQ ID NO: 50); and a CDR-H3 comprising DFNYYVGAMDX₂₀ (SEQ ID NO: 51); and
   a light chain variable (VL) region, wherein the VL region comprises a CDR-L1 comprising X₂₁SX₂₂QSLX₂₃HSNGNTYX₂₄H (SEQ ID NO: 52); a CDR-L2 comprising X₂₅VSNRFS (SEQ ID NO: 53); and a CDR-L3 comprising SQSX₂₆X₂₇X₂₈WT (SEQ ID NO: 54);
   and wherein X, to X₂₈ includes any amino acid; optionally wherein X₁ includes a polar amino acid with an uncharged or a positively charged side chain; X₂ includes a nonpolar amino acid with an aliphatic side chain; X₃ includes a polar amino acid with an uncharged or a negatively charged side chain; X₄ includes a nonpolar amino acid with an aliphatic side chain; X₅ includes a polar amino acid with an uncharged side chain; X₆ includes a polar amino acid with a negatively charged or an uncharged side chain; X₇ includes a nonpolar amino acid with a aliphatic side chain; X₈ includes a nonpolar amino acid with an aliphatic or aromatic side chain; X₉ includes a polar amino acid with an uncharged side chain; X₁₀ includes a polar amino acid with a negatively charged side chain or a nonpolar amino acid with an aliphatic side chain; X₁₁ includes an nonpolar amino acid with an aliphatic side chain or a polar amino acid with a positively charged side chain; X₁₂ includes a polar amino acid with uncharged side chain or a nonpolar amino acid with an aliphatic side chain; X₁₃ includes a nonpolar amino acid with an aromatic side chain; X₁₄ includes a polar amino acid with uncharged side chain; X₁₅ includes a polar amino acid with a negatively charge side chain; X₁₆ includes a polar amino acid with positively charged or uncharged side chain; X₁₇ includes a nonpolar amino acid with an aliphatic or aromatic side chain; X₁₈ includes a polar amino acid with a positively charged or an uncharged side chain; X₁₉ includes a polar amino acid with a negatively charged side chain or a nonpolar amino acid with an aliphatic side chain; X₂₀ includes a nonpolar amino acid with an aromatic side chain; X₂₁ includes a polar amino acid with a positively charged side chain; X₂₂ includes a polar amino acid with an uncharged side chain; X₂₃ includes a nonpolar amino acid with an aliphatic side chain; X₂₄ includes a nonpolar amino acid with an aliphatic side chain; X₂₅ includes a polar amino acid with a positively charged or an uncharged side chain; X₂₆ includes a polar amino acid with an uncharged side chain; X₂₇ includes a polar amino acid with a positively charged side chain or nonpolar amino acid with an aromatic side chain; and X₂₈ includes a nonpolar amino acid with an aliphatic side chain.
5. The agent of any one of embodiments 1-4, wherein X, is S or R; X₂ is V or I; X₃ is D or N; X₄ is I or M; X₅ is N or S; X₆ is D, E, or N; X₇ is I or M; X₈ is I, M, or F; X₉ is T or N; X₁₀ is D or G; X₁₁ is G or H; X₁₂ is P, S, or A; X₁₃ is Y or F; X₁₄ is S or N; X₁₅ is D or E; X₁₆ is K or T; X₁₇ is I, A, or F; X₁₈ is K, R, or T; X₁, is D or G; X₂₀ is F or Y; X₂₁ is R or K; X₂₂ is S or T; X₂₃ is L, V, or I; and X₂₄ is L or V; X₂₅ is K or Q; X₂₆ is T or S; X₂₇ is H or Y; and X₂₈ is I or V.
6. The agent of any one of embodiments 1-5, wherein the agent comprises an anti-TREML antibody or antigen-binding fragment thereof comprising
   (i) a heavy chain variable (VH) region, wherein the VH region comprises a heavy chain complementarity determining region 1 (CDR-H1) comprising DYGMA (SEQ ID NO: 20); a CDR-H2 comprising FISNLAYSVYYADTVTG (SEQ ID NO: 21); and a CDR-H3 comprising EDYGDNGAIDY (SEQ ID NO: 22); and a light chain variable (VL) region, wherein the VL region comprises a light chain complementarity region 1 (CDR-L1) comprising RSSQNIVHSNGNTYLE (SEQ ID NO: 23); a CDR-L2 comprising KVSNRFS (SEQ ID NO: 24); and a CDR-L3 comprising FQGSHVPPT (SEQ ID NO: 25);
   (ii) a heavy chain variable (VH) region, wherein the VH region comprises a heavy chain complementarity determining region 1 (CDR-H1) comprising DYGMA (SEQ ID NO: 20); a CDR-H2 comprising FISNLAYSVYYADTVTG (SEQ ID NO: 21); and a CDR-H3 comprising EDYGDNGAIDY (SEQ ID NO: 22); and a light chain variable (VL) region, wherein the VL region comprises a light chain complementarity region 1 (CDR-L1) comprising RSSQSIVHSNGNTYLE (SEQ ID NO: 26); a CDR-L2 comprising KVSNRFS (SEQ ID NO: 24); and a CDR-L3 comprising FQGSHVPPT (SEQ ID NO: 25);
   (iii) a heavy chain variable (VH) region, wherein the VH region comprises a heavy chain complementarity determining region 1 (CDR-H1) comprising DYGMA (SEQ ID NO: 20); a CDR-H2 comprising FISNLAYRIYYADTVTG (SEQ ID NO: 27); and a CDR-H3 comprising EDYGNNGAMDY (SEQ ID NO: 28); and a light chain variable (VL) region, wherein the VL region comprises a light chain complementarity region 1 (CDR-L1) comprising RSSQSIVHSNGNTYLE (SEQ ID NO: 26); a CDR-L2 comprising KVSNRFS (SEQ ID NO: 24); and a CDR-L3 comprising FQGSHVPPT (SEQ ID NO: 25);
   (iv) a heavy chain variable (VH) region, wherein the VH region comprises a CDR-H1 comprising DYVIH (SEQ ID NO: 32); a CDR-H2 comprising YMNPYTDGPKYSDKIKD (SEQ ID NO: 33); and a CDR-H3 comprising DFNYYVGAMDF (SEQ ID NO: 34); and a light chain variable (VL) region, wherein the VL region comprises a CDR-L1 comprising RSSQSLLHSNGNTYLH (SEQ ID NO: 35); a CDR-L2 comprising KVSNRFS (SEQ ID NO: 24); and a CDR-L3 comprising SQSTHIWT (SEQ ID NO: 36);
   (v) a heavy chain variable (VH) region, wherein the VH region comprises a CDR-H1 comprising DYVIH (SEQ ID NO: 32); a CDR-H2 comprising YINPYTGGPKYSETARG (SEQ ID NO: 37); and a CDR-H3 comprising DFNYYVGAMDF (SEQ ID NO: 34); and a light chain variable (VL) region, wherein the VL region comprises a CDR-L1 comprising RSTQSLVHSNGNTYVH (SEQ ID NO: 38); a CDR-L2 comprising KVSNRFS (SEQ ID NO: 24); and a CDR-L3 comprising SQSTYVWT (SEQ ID NO: 56);
   (vi) a heavy chain variable (VH) region, wherein the VH region comprises a CDR-H1 comprising EYVIH (SEQ ID NO: 39); a CDR-H2 comprising YFNPYTGGSKFNEKFKD (SEQ ID NO: 40); and a CDR-H3 comprising DFNYYVGAMDY (SEQ ID NO: 55); and a light chain variable (VL) region, wherein the VL region comprises a CDR-L1 comprising RSSQSLVHSNGNTYLH (SEQ ID NO: 41); a CDR-L2 comprising KVSNRFS (SEQ ID NO: 24); and a CDR-L3 comprising SQSSHIWT (SEQ ID NO: 42);
   (vii) a heavy chain variable (VH) region, wherein the VH region comprises a CDR-H1 comprising NYVMH (SEQ ID NO: 43); a CDR-H2 comprising YFNPYNGHAKYSEKFTG (SEQ ID NO: 44); and a CDR-H3 comprising DFNYYVGAMDY (SEQ ID NO: 55); and a light chain variable (VL) region, wherein the VL region comprises a CDR-L1 comprising RSSQSLIHSNGNTYLH (SEQ ID NO: 45); a CDR-L2 comprising QVSKRFS (SEQ ID NO: 46); and a CDR-L3 comprising SQSTHIWT (SEQ ID NO: 36); or
   (viii) a heavy chain variable (VH) region, wherein the VH region comprises a CDR-H1 comprising DYVIH (SEQ ID NO: 32); a CDR-H2 comprising YINPYTGGPKYSETAKG (SEQ ID NO: 47); and a CDR-H3 comprising DFNYYVGAMDF (SEQ ID NO: 34); and a light chain variable (VL) region, wherein the VL region comprises a CDR-L1 comprising KSTQSLVHSNGNTYVH (SEQ ID NO: 48); a CDR-L2 comprising KVSNRFS (SEQ ID NO: 24); and a CDR-L3 comprising SQSTYVWT (SEQ ID NO: 56).
7. The agent of any one of embodiments 1-6, wherein the agent comprises
   (i) a VH region comprising SEQ ID NO: 4 and a VL region comprising SEQ ID NO: 5;
   (ii) a VH region comprising SEQ ID NO: 6 and a VL region comprising SEQ ID NO: 7;
   (iii) a VH region comprising SEQ ID NO: 8 and a VL region comprising SEQ ID NO: 9;
   (iv) a VH region comprising SEQ ID NO: 10 and a VL region comprising SEQ ID NO: 11;
   (v) a VH region comprising SEQ ID NO: 12 and a VL region comprising SEQ ID NO: 13;
   (vi) a VH region comprising SEQ ID NO: 14 and a VL region comprising SEQ ID NO: 15;
   (vii) a VH region comprising SEQ ID NO: 16 and a VL region comprising SEQ ID NO: 17,
   (viii) a VH region comprising SEQ ID NO: 18 and a VL region comprising SEQ ID NO: 19.
8. A method of treating or alleviating one or more symptoms of a disease or condition associated with or characterized by immune suppression in a subject in need thereof, the method comprising administering one or more agents of any one of embodiments 1-7 to the subject, and reversing immune suppression in the subject, thereby treating or alleviating one or more symptoms of the disease or condition.
9. The method of embodiment 8, wherein reversing immune suppression comprises inhibiting an inflammatory response including inhibiting the release of cytokines such as IL-10 or inducing the release of cytokines such as TNF-α, or wherein the immune suppression comprises inhibiting inflammation.
10. The method of embodiment 8 or 9, wherein the inflammatory response can be a monocyte-mediated, macrophage-mediated, and/or neutrophil-mediated inflammatory response.
11. The method of any one of embodiments 8-10, wherein the disease or condition includes the growth of a malignant tumor, tumor angiogenesis, cancer, chronic infection, sepsis, immune exhaustion, or immunosenescence in aging.
12. The method of any one of embodiments 8-11, wherein the cancer includes melanoma, lung cancer, squamous cell carcinomas of the lung, head and neck cancer, breast cancer, ovarian cancer, uterine cancer, prostate cancer, gastric carcinoma, cervical cancer, esophageal carcinoma, bladder cancer, kidney cancer, brain cancer, liver cancer, colon cancer, bone cancer, pancreatic cancer, skin cancer, cutaneous or intraocular malignant melanoma, ovarian cancer, rectal cancer, cancer of the anal region, stomach cancer, testicular cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, esophagus cancer, small intestine cancer, endocrine system cancer, thyroid gland cancer, parathyroid gland cancer, adrenal gland cancer, sarcoma of soft tissue, urethra cancer, penis cancer, chronic or acute leukemia, solid tumors of childhood, lymphocytic lymphoma, carcinoma of the renal pelvis, neoplasm of the central nervous system (CNS), primary CNS lymphoma, spinal axis tumor, brain stem glioma, pituitary adenoma, Kaposi's sarcoma, epidermoid cancer, squamous cell cancer, T cell lymphoma, and gastrointestinal tract cancer, and optionally wherein the cancer comprises colon cancer.
13. The method of embodiment 12, wherein the chronic or acute leukemia is acute myeloid leukemia, chronic myeloid leukemia, acute lymphoblastic leukemia, or chronic lymphocytic leukemia.
14. The method of any one of embodiments 8-12, wherein the method inhibits the growth of a malignant tumor or cancer cells.
15. A method of inhibiting the binding of TREML1 to CD11b, wherein the method includes administering one or more agents of any one of embodiments 1-7 to cells expressing CD11b and are in the presence of TREML1, thereby inhibiting the binding of TREML1 to CD11b.
16. A method of reversing immune suppression in cells, wherein the method includes administering one or more agents of any one of embodiments 1-7 to cells induced by TREML1 binding I-domain of CD11 b to be immune suppressive.
17. A method of inhibiting PD-L1 expression on cells, wherein the method includes administering one or more agents of any one of embodiments 1-7 to cells induced by TREML1 binding I-domain of CD11 b to express PD-1.
18. The method of any one of embodiments 15-17, wherein the cells include immune cells.
19. The method of any one of embodiments 15-18, wherein the cells include cells of the innate immune response system.
20. The method of any one of embodiments 15-19, wherein the cells include macrophages, neutrophils, monocytes, dendritic cells, natural killer cells, granulocytes, or a combination thereof.
21. The method of any one of embodiments 15-20, wherein the cells are in a subject, and optionally wherein the subject is a mammal.
22. The method of any one of embodiments 15-21, wherein the method reverses the immune suppression of the cells and/or treats a disease or condition associated with immune suppression.
23. A method of diagnosing cancer or detecting the presence of a malignant tumor in a subject, wherein the method includes obtaining a biological sample from the subject, and detecting TREML1 in the sample, thereby detecting cancer or tumor in the subject.
24. A method of determining whether a subject would be responsive to all therapies that can be administered as a form of cancer treatment, wherein the method includes obtaining a biological sample from the subject, and detecting presence of TREML1 in the sample, thereby determining the subject would be responsive to all therapies that can be administered as a form of cancer treatment.
25. The method of embodiments 23 or 24, wherein the method further includes quantifying the expression of TREML1 in the sample and optionally, wherein the method further includes comparing the expression of TREML1 in the subject to the expression of TREML1 of a control. In embodiments, the control is a healthy subject. In embodiments, the control is a reference value associated with expression of TREML1 in a healthy subject.
26. The method of any one of embodiments 23-25, wherein the biological sample includes body fluids, cells, or tissues, and optionally wherein body fluids include blood, urine, saliva, bile, bone marrow aspirate, breast milk, cerebral spinal fluid (CSF), plasma, serum, stool, vaginal fluid, or synovial fluid; optionally wherein cells include blood cells, epithelial cells, fibroblasts, hepatocytes, immune cells (such as T cells, B cells, NK cells, monocytes, macrophages, dendritic cells and the like), stem cells, peripheral blood cells, or stem cells; and optionally, wherein the tissue is from a biopsy or a resected tumor.
27. The method of any one of embodiments 23-26, wherein detecting the presence of TREML1 includes performing an immunological assay, a histological assay, a cytological assay, an enzyme-linked immunosorbent assay (ELISA), a bead-based detection assay, a DNA or RNA expression assay or an aptamer-based assay.
28. The method of any one of embodiments 23-27, wherein detecting the presence of TREML1 includes using the agent of any one of embodiments 1-7 to detect the presence of TREML1.
29. The method of any one of embodiments 23-28, wherein the cancer or malignant tumor includes a cancer of embodiment 12 or 13.
30. The method of any one of embodiments 23-29, wherein the method further includes treating the subject with ICB therapy, and optionally wherein ICB therapy includes administering atezolizumab, avelumab, or durvalumab to the subject.
31. The method of any one of embodiments 23-30, wherein the method further includes treating the subject by administering one or more agents of any one of embodiments 1-7.
32. A composition including one or more agents of any one of embodiments 1-7, and optionally wherein the composition further includes a carrier.
33. The composition of embodiment 32, wherein the composition is a pharmaceutical composition, and optionally wherein the carrier is a pharmaceutically acceptable carrier.
34. A kit including one or more agents of any one of embodiments 1-7 or a pharmaceutical composition of embodiment 33 for diagnosing cancer, detecting the presence of malignant tumor and/or determining whether a subject would be responsive to all therapies that can be administered as a form of cancer treatment.
35. A kit including one or more agents of any one of embodiments 1-7 or a pharmaceutical composition of embodiment 33 for treating a subject, and optionally wherein the kit includes a device for administering the agent to the subject.
36. The kit of embodiment 35, wherein the kit further includes one or more therapies that can be administered as a form of cancer treatment, and optionally wherein the kit includes means for administering one or more therapies that can be administered as a form of cancer treatment to the subject.

### EXAMPLES

### Materials and Methods

### Cell Culture and Stable Transfection

Stable transfection of human or mouse TREML1 in HEK293 cells (BCRC) was performed using jetPRIME (PolyPlus) transfection protocols. Briefly, HEK293 cells were cultured in Dulbecco's Modified Eagle's Medium (DMEM, Corning), supplemented with 10% heat-inactivated fetal bovine serum (Gibco) and 50 IU/mL penicillin and streptomycin (Corning) at 37° C. The cells were seeded at 8×10⁵ cells/well on a 6-well plate (Coster). The next day, a mixture of the jetPRIME reagent and 2 μg pcDNA 3.1/human TREML1 (GenScript) or pcDNA 3.1/mouse TREML1 (GenScript) expression plasmid carrying a neomycin-resistance gene was added to the cells, and the cells were cultured for 24 hr. Then, the selection antibiotic, G418 (InVivoGen), was added at a concentration of 1 mg/ml, and half of the culture media containing the antibiotic were changed every 2 to 3 days. After 3 weeks, the human or mouse TREML1-expression cells were collected using the Cell Sorter (SH800Z, SONY) to pick up human or mouse TREML1-high expression cells and seeded at single cell/well and 5000 cells/well on a 24-well plate (Coster) and a 6-well plate. Cells were maintained in DMEM medium with 10% heat-inactivated fetal bovine serum, 50 IU/mL penicillin and streptomycin, and 1 mg/ml G418 at 37° C. After the cells were enriched, human or mouse TREML1 expression was analyzed with anti-human TREML1/TLT-1 (MAB2394, R&D systems) or anti-mouse TREML1/TLT-1 (MAB24241, R&D systems) antibody by flow cytometry. Finally, the stable clones of HEK293/human TREML1 and HEK293/mouse TREML1 were picked up.

### Flow Cytometry

The HEK293/human TREML1 or HEK293/mouse TREML1 were counted and washed twice with staining buffer (PBS containing 1% FBS and 0.1% sodium azide). Cells were adjusted at a concentration of 1×10⁵ cells/ml in staining buffer and treated with 10 μg/ml of the anti-TREML1 antibodies, or isotype control (MOPC21). The cells were then incubated at 37° C. for 30 min. After washing with the staining buffer, cells were treated with APC conjugated anti-mouse IgG antibodies for 15 min. After washing with the staining buffer, the cells were analyzed by flow cytometry.

### Solid Phase Binding Assay

To investigate the TREML1/I-domain of CD11 b interaction, 5 μg/ml of recombinant I-domain of CD11 b (SEQ ID: 1) and BSA (negative control) proteins were coated separately on 96 well plates and incubated overnight at 4° C. Plates were added the blocking buffer (2% BSA in PBS) to avoid the nonspecific binding for 2 hours (hrs) at room temperature. A serial diluted TREML1 ECD was added and allowed to incubate at room temperature for 2 hrs. 1ug/ml of anti-TREML1 antibody (MAB2394, R&D systems) was added for 1 hr. Next, the secondary antibody with HRP labeling, anti-Rat IgG-HRP, was added to the plate and incubated for 30 minutes at room temperature. Plates were washed thrice with 0.05% tween 20 PBS buffer (PBST) between each step. Finally, TMB substrate was added and incubated for 10-15 min to detect HRP labeling antibody. After that, a stop solution of 1N HCL was added to stop the reaction. Absorbance at OD450/540 nm was measured by a microplate reader. To measure the half-maximal saturation binding of anti-TREML1 antibodies to TREML1, 5 μg/ml of human TREML1 ECD (SEQ ID: 2) or mouse TREML1 ECD (SEQ ID: 3) were coated on 96 well plates and incubated overnight at 4° C. Plates were added the blocking buffer (2% BSA in PBS) to avoid the nonspecific binding for 2 hr at room temperature. A serial diluted anti-TREML1 antibody was added and allowed to incubate at room temperature for 2 hr. Next, the secondary antibody with HRP labeling, anti-mouse IgG-HRP, was added to the plate and incubated for 30 minutes at room temperature. Plates were washed three times with 0.05% tween20 PBS buffer (PBST) between each step. Finally, TMB substrate was added and incubated for 10-15 min to detect HRP labeling antibody. After that, a stop solution of 1N HCL was added to stop the reaction. Absorbance at OD450/540 nm was measured by a microplate reader.

### Preparation of Human White Blood Cells (WBCs) and Human Monocytes

Peripheral blood samples f were collected from healthy volunteer donors by venipuncture and bleeding into ACD vacutainer tubes. Human white blood cells (WBCs) were separated from peripheral blood using hypotonic erythrocyte lysis in ammonium chloride containing ACK solution. Human monocytes were isolated from human peripheral blood mononuclear cells, which were separated from whole blood through Ficoll-Paque density gradient centrifugation. The selection was carried out with a positive CD14 isolation kit (Miltenyi Biotec).

### TREML-1 Immunostaining Protocol and Scoring

Human tumor tissue arrays were stained with an anti-TREML1 antibody (26A6) using a concentration of 1 mg/ml (1:100, 60 minutes at room temperature), with mouse Probe DAB Brown (30 min at room temperature) and double-stained CD68 macrophage marker (labeled with green color) by Mouse/Rabbit Double Stain Kit (With DAB Brown/HRP Green) (BioTnA, TADSO3) detection kit technology. The signal visualization was performed by diaminobenzidine (DAB) staining, and sections were counter-stained with hematoxylin. TREML1 expression was scored by the positive percentage of total area, and H-score indicated the TREML1 intensities by calculating the sum of the percentages at all positive staining intensities.

### In Vivo TREML1 Detection

Anti-TREML1 antibody (26A6) or isotype control (MOPC21) antibodies were labeled with VivoTag 680XL (PerkinElmer) according to the manufacturer's instructions. C57BL/6 mice were injected with MC38 colon cancer cells subcutaneously. After tumor volumes were >300 mm³, 10 μg of VivoTag 680XL-conjugated anti-TREML1 antibody (26A6) and isotype control (MOPC21) were injected subcutaneously and imaged at various time points using the IVIS Spectrum and FMT instrument. Images shown are at 24 hours post-injection of the antibody conjugate.

### Protocol For Cancer Treatment

C57BL/6 mice were inoculated subcutaneously with 3×10⁵ MC38 cells. Treatment was started 11 days after tumor inoculation. Tumor-bearing mice were treated intraperitoneally with 10 mg/kg of anti-TREML1 antibodies twice per week. Mice were monitored and scored twice weekly for the formation of palpable tumors and sacrificed if tumors exceeded the predetermined size of 2,000 mm³. Tumor volumes were measured with calipers and calculated with the following formula: A×B×B×0.52, where A is the largest diameter, and B is the smallest diameter.

### Example 1: I-Domain of CD11b is the Receptor for TREML1, and Blocking TREML1 and CD11b Interaction can Reverse TREML1-Induced Immunosuppression

Soluble TREML1 was previously reported to directly bind innate immune cells and trigger cells to express immunosuppressive phenotypes (WO2016197975A1). To screen for the potential immune suppressive receptor of TREML1 on the monocyte membrane, a recombinant histidine-tagged TREML1 ECD bound to Ni-column was used. Monocyte lysates were then incubated with TREML1 ECD-bound column. TREML1 ECD binding complexes were eluted and then separated by SDS-PAGE. Following the trypsin digestion of the TREML1 ECD binding complex, digested peptides were determined on LC-MS/MS. Interpretation of the MS/MS spectra was performed by the MASCOT database. According to MASCOT probability analysis, some immune suppressive receptors (such as CD11b, CD18, CD33 (Siglec-3), CD329 (Siglec-9), and HSP90b) have higher scores than other protein candidates. CD11 b was selected as the representative immune suppressive receptor for further studies.

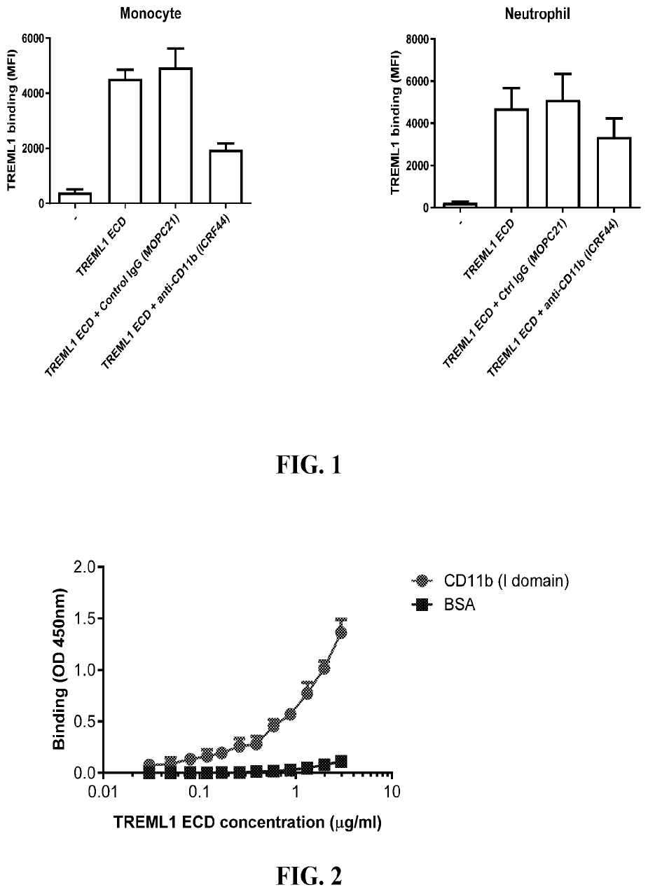

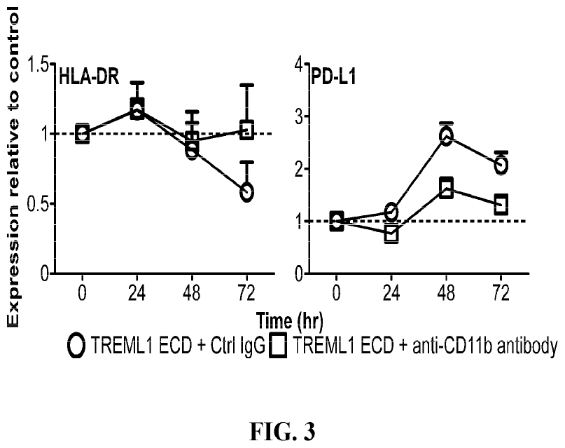

To examine whether soluble TREML1 binds CD11b+ immune cells, the binding of TREML1 ECD to human monocytes and neutrophils in the presence of anti-CD11 b antibody (ICRF44) was determined by flow cytometry. As shown in FIG. 1, a CD11 b specific antibody can compete with TREML1 ECD for binding monocytes and neutrophils expressing CD11b+ on the cell surface. A solid phase binding study showed that TREML1 ECD binds to the I-domain of CD11 b in a concentration-dependent manner (FIG. 2). Treatment with anti-CD11 b antibody (ICRF44) normalized the levels of HLA-DR expression. It reduced the levels of PD-L1 expression on TREML1 ECD-treated monocytes (FIG. 3). These results showed that CD11 b participates in TREML1-induced immune suppression. Blocking CD11b and TREML1 interaction can reverse TREML1-induced immune response.

### Example 2: Anti-TREML1 Antibodies Specifically Bind to TREML1 ECD and Block TREML1 ECD Binding to CD11b+ Immune Cells

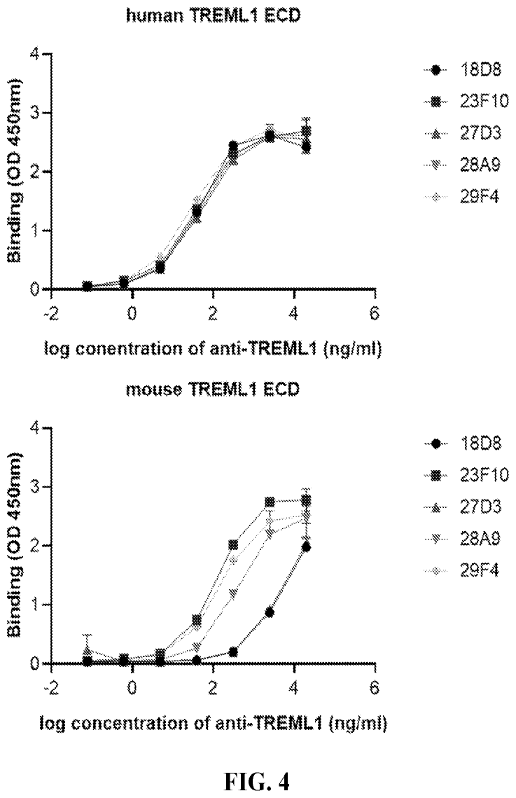

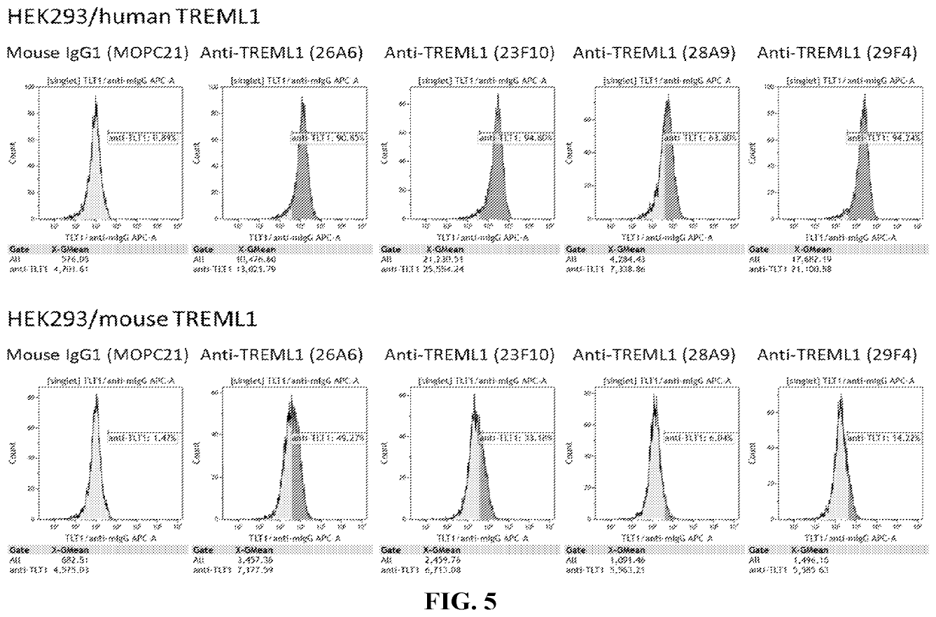

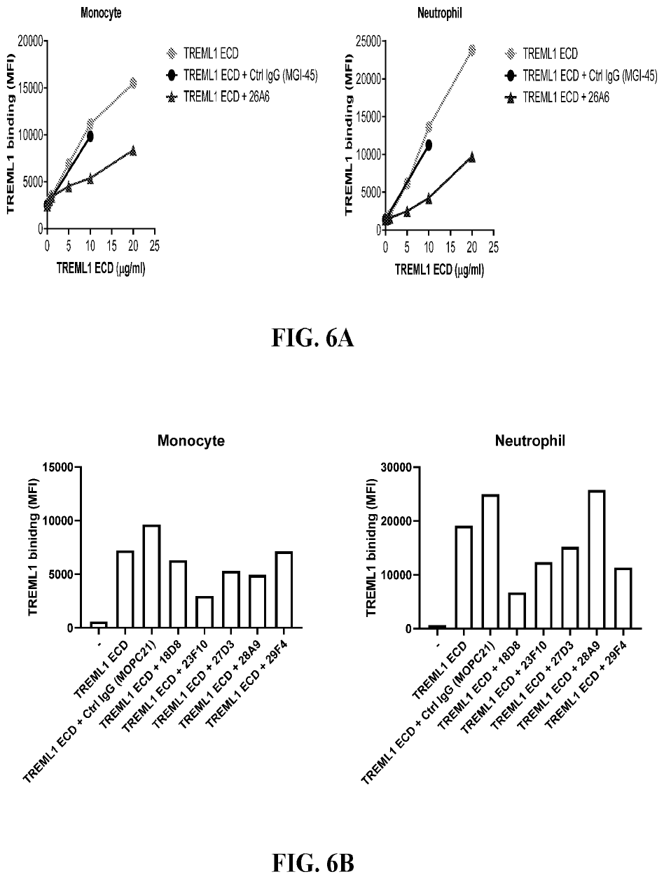

Several anti-TREML1 antibodies capable of binding human and murine TREML1 ECD were generated. As shown in FIG. 4, anti-TREML1 antibodies concentration-dependently bind human and mouse TREML1 ECD in the solid phase binding assay. Half-maximal saturation binding of anti-TREML1 antibodies to human TREML1 ECD and mouse TREML1 ECD are shown in Table 4. VH and VL regions of these TREML1 antibodies are listed in Table 3. These TREML1-binding antibodies (17E6, 25C6, and 26A6) exhibit high levels of paratope residue conservation across all CDR regions (Table 1). In contrast, CDR-H1, CDR-H3, CDR-L1, and CDR-L2 of (18D8, 23F10, 27D3, 28A9, and 29F4) seem to retain a greater level of germline conservation than CDR-H2 and CDR-L3 (Table 2). To examine whether these anti-TREML1 antibodies can bind TREML1 on the cell surface, HEK293 cells, which do not express endogenous TREML1, were transfected with pcDNA3.1/human TREML1 and pcDNA3.1/mouse TREML1 plasmids using liposome transfections. After G418 selections, several single-cell clones stably expressing the human and mouse TREML1 on the cell surface were obtained. Compared with isotype control antibody (MOPC21), anti-TREML1 antibodies (26A6, 23F10, 28A9, and 29F4) can bind to human TREML1 on the HEK293/human TREML1 (FIG. 5, upper). In addition, clones 26A6, 23F10, and 29F4 can further cross-react with mouse TREML1 on the HEK293/mouse TREML1 (FIG. 5 lower). To examine whether anti-TREML1 antibody can block TREML1 ECD binding to CD11 b+ immune cells, the binding of TREML1 ECD to human monocytes and neutrophils in the presence of anti-TREML1 antibody was determined by flow cytometry. As shown in FIG. 6, using TREML1 specific antibody can reduce TREML1 ECD binding to neutrophils and monocytes that express CD11 b+ on the cell surface.

TABLE 4
Half-Maximal Binding of Anti-TREM1 Antibodies
to Human and Mouse TREML1 ECD

| Antibody | Antigen | EC50 (nM) |
|---|---|---|
| 26A6 | Human TREML1 ECD | 0.32 |
| 26A6 | Mouse TREML1 ECD | 0.4 |
| 18D8 | Human TREML1 ECD | 0.25 |
| 18D8 | Mouse TREML1 ECD | 33.4 |
| 23F10 | Human TREML1 ECD | 0.27 |
| 23F10 | Mouse TREML1 ECD | 0.82 |
| 27D3 | Human TREML1 ECD | 0.31 |
| 27D3 | Mouse TREML1 ECD | 24.95 |
| 28A9 | Human TREML1 ECD | 0.30 |
| 28A9 | Mouse TREML1 ECD | 2.48 |
| 29F4 | Human TREML1 ECD | 0.18 |
| 29F4 | Mouse TREML1 ECD | 0.94 |

### Example 3: Treatment with Anti-TREML1 Antibody can Significantly Reduce PD-L1 Expression Induced by TREML1 ECD

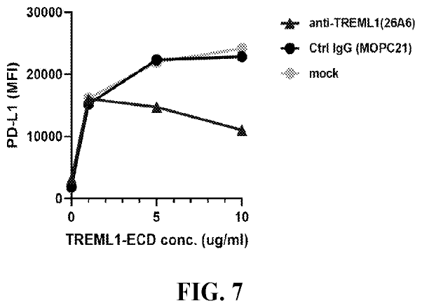

To examine whether anti-TREML1 antibody can reverse TREML1 ECD-mediated cellular suppression, human WBCs were treated with/without 10 μg/ml of anti-TREML1 antibody (26A6) in the presence of human TREML1 ECD at 1, 5, 10 μg/ml for 24 hours and the PD-L1 expression were analyzed by flow cytometry. As shown in FIG. 7, the PD-L1 expression in human monocytes increased after incubation with TREML1 ECD at 1, 5, and 10 μg/ml for 24 hrs, and reduced in the presence of anti-TREML1 antibody.

### Example 4: TREML1 is Enriched in the Tumor Microenvironment

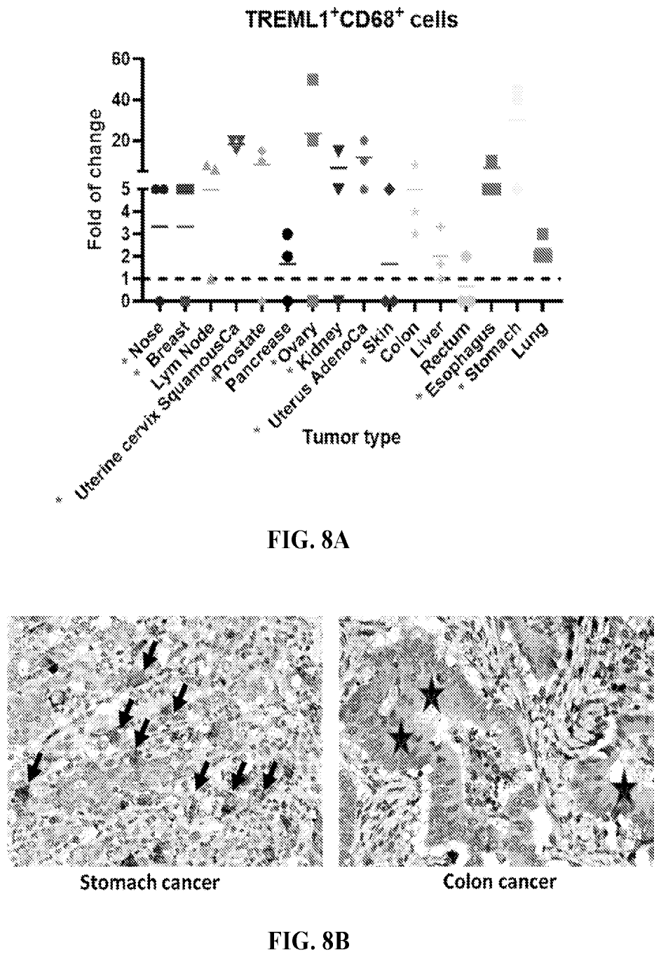

To examine whether platelet-derived TREML1 is enriched in the tumor, an anti-TREML1 antibody (26A6) was used to detect TREML1 accumulation in human cancer biopsy. The TREML1 expression in human tissue was examined by immunostaining in formalin-fixed paraffin-embedded tissue, and the intensity of TREML1 expression in most of the human tumors was significantly higher than in normal tissue (FIG. 8A). The percentage of TREML-1-bound macrophages were quantitated, and the fold of change was calculated by dividing the percentage of TREML1/CD68 double positive cells in tumor tissues by that in normal tissue adjacent to the tumor (NAT). Since zero percentage of TREML1/CD68 double positive cells was found in some normal tissue, including Nose, Breast, Uterus, Prostate, Ovary, Kidney, Skin, Esophagus, and Stomach (marked with asterisk in FIG. 8A), the percentage of one, instead of zero, was used for calculating the fold of change.

Since soluble TREML1 can be secreted by activated platelet and bound to immune cells, TREML1 (Brown color) can be found colocalized with macrophages (Green color) and also distributed around the tumor stromal region (FIG. 8B). In FIG. 8B, the TREML1 (Brown color) can be found colocalized with macrophages (Green color) and distributed around the tumor stromal region. The representative picture of human stomach cancer tissue shows the site of TREML1 and macrophage colocalization (marked by arrows) and the representative picture of colon cancer shows TREML1 in the tumor (marked by an asterisk).

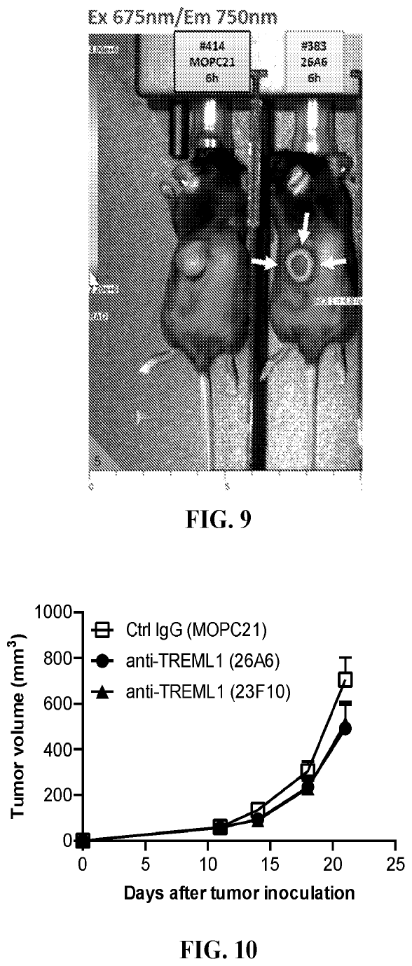

To examine whether TREML1 was present in the tumor microenvironment of mice, mice were subcutaneously injected with MC38 colon cancer. Anti-TREML1 antibody (26A6) conjugated VivoTag 680XL (PerkinElmer) was used to detect TREML1 accumulation in vivo. Using the IVIS image, TREML1 was observed to be highly enriched in the MC38 colon cancer (FIG. 9). Collectively, these data suggest that TREML1 is highly increased in the tumor microenvironment. TREML1 can serve as a prognostic marker and as a potential target for cancer therapy.

### Example 5: Treatment with Anti-TREML1 Antibody can Significantly Reduce Tumor Growth

To examine the effect of TREML1 blockade on antitumor immunity, the anti-TREML1 antibody was tested as monotherapy in the MC38 colon cancer model. Mice were subcutaneously injected with MC38 colon cancer cells at Day 0. When tumor volumes were approximately 30-100 mm³, mice were injected intraperitoneally (ip) with either control IgG (MOPC21; 10 mg/kg), anti-TREML1 (26A6; 10 mg/kg), oranti-TREML1 (23F10; 10 mg/kg) antibody. Injections were repeated every three to four days. Efficiency was determined by monitoring tumor volumes for each group. As shown in FIG. 10, TREML1 blockade with anti-TREML1 antibody potently delayed the subcutaneous growth of MC38 tumors.

### Example 6: Identification of Cancer Patients for Cancer Therapy

Biopsies are obtained from patients diagnosed with cancer. Immunohistochemical staining is performed on the biopsy samples using anti-TREML1 antibody. The intensity and percentage of the staining are quantitated and compared with the staining in corresponding control (normal or healthy) tissue. Patients with a high level of TREML1 as compared to control are identified as being candidates for cancer therapy.

The subject matter described above is provided by way of illustration only and should not be construed as limiting. Various modifications and changes may be made to the subject matter described herein without following the example embodiments and applications illustrated and described, and without departing from the true spirit and scope of the present disclosure, which is set forth in the following claims.

All publications, patents, and patent applications cited in this specification are incorporated herein by reference in their entireties as if each individual publication, patent, or patent application were specifically and individually indicated to be incorporated by reference. While the foregoing has been described in terms of various embodiments, the skilled artisan will appreciate that various modifications, substitutions, omissions, and changes may be made without departing from the spirit thereof.

1. An agent that inhibits the binding of triggering receptor expressed by myeloid cell (TREM)-like transcript-1 (TREML1) to CD11 b, wherein the agent is an antibody or antigen-binding fragment thereof.

2. The agent of claim 1, wherein the CD11b is expressed on immune cells.

3. The agent of claim 1, wherein the agent is a polyclonal antibody, a monoclonal antibody, a humanized antibody, a chimeric antibody, a bispecific antibody, or an antigen-binding fragment thereof.

4. The agent of claim 1, wherein the agent comprises an anti-TREML antibody or antigen-binding fragment thereof comprising
i) a heavy chain variable (VH) region, wherein the VH region comprises a heavy chain complementarity determining region 1 (CDR-H1) comprising DYGMA (SEQ ID NO: 20); a CDR-H2 comprising FISNLAYX₁X₂YYADTVTG (SEQ ID NO: 29); and a CDR-H3 comprising EDYGX₃NGAX₄DY (SEQ ID NO: 30); and
a light chain variable (VL) region, wherein the VL region comprises a light chain complementarity region 1 (CDR-L1) comprising RSSQX₅IVHSNGNTYLE (SEQ ID NO: 31); a CDR-L2 comprising KVSNRFS (SEQ ID NO: 24); and a CDR-L3 comprising FQGSHVPPT (SEQ ID NO: 25);
or
ii) a heavy chain variable (VH) region, wherein the VH region comprises a CDR-H1 comprising X₆YVX₇H (SEQ ID NO: 49); a CDR-H2 comprising YX₈NPYX₉X₁₀X₁₁X₁₂KX₁₃X₁₄X₁₅X₁₆X₁₇X₁₈X₁₉ (SEQ ID NO: 50); and a CDR-H3 comprising DFNYYVGAMDX₂₀ (SEQ ID NO: 51); and
a light chain variable (VL) region, wherein the VL region comprises a CDR-L1 comprising X₂₁SX₂₂QSLX₂₃HSNGNTYX₂₄H (SEQ ID NO: 52); a CDR-L2 comprising X₂₅VSNRFS (SEQ ID NO: 53); and a CDR-L3 comprising SQSX₂₆X₂₇X₂₈WT (SEQ ID NO: 54);
and wherein X₁ to X₂₈ is any amino acid; optionally wherein
X₁ is a polar amino acid with an uncharged or a positively charged side chain; X₂ is a nonpolar amino acid with an aliphatic side chain; X₃ is a polar amino acid with an uncharged or a negatively charged side chain; X₄ is a nonpolar amino acid with an aliphatic side chain; X₅ is a polar amino acid with an uncharged side chain; X₆ a polar amino acid with a negatively charged or an uncharged side chain; X₇ is a nonpolar amino acid with a aliphatic side chain; X₈ is a nonpolar amino acid with an aliphatic or aromatic side chain; X₉ is a polar amino acid with an uncharged side chain; X₁₀ is a polar amino acid with a negatively charged side chain or a nonpolar amino acid with an aliphatic side chain; X₁₁ is a nonpolar amino acid with an aliphatic side chain or a polar amino acid with a positively charged side chain; X₁₂ is a polar amino acid with uncharged side chain or a nonpolar amino acid with an aliphatic side chain; X₁₃ is a nonpolar amino acid with an aromatic side chain; X₁₄ is a polar amino acid with uncharged side chain; X₁₅ is a polar amino acid with a negatively charge side chain; X₁₆ is a polar amino acid with positively charged or uncharged side chain; X₁₇ is a nonpolar amino acid with an aliphatic or aromatic side chain; X₁₈ is a polar amino acid with a positively charged or an uncharged side chain; X₁₉ is a polar amino acid with a negatively charged side chain or a nonpolar amino acid with an aliphatic side chain; X₂₀ is a nonpolar amino acid with an aromatic side chain; X₂₁ is a polar amino acid with a positively charged side chain; X₂₂ is a polar amino acid with an uncharged side chain; X₂₃ is a nonpolar amino acid with an aliphatic side chain; X₂₄ is a nonpolar amino acid with an aliphatic side chain; X₂₅ is a polar amino acid with a positively charged or an uncharged side chain; X₂₆ is a polar amino acid with an uncharged side chain; X₂₇ is a polar amino acid with a positively charged side chain or nonpolar amino acid with an aromatic side chain; and X₂₈ is a nonpolar amino acid with an aliphatic side chain.

5. The agent of claim 4, wherein X₁ is S or R; X₂ is V or I; X₃ is D or N; X₄ is I or M; X₅ is N or S; X₆ is D, E, or N; X₇ is I or M; X₈ is I, M, or F; X₉ is T or N; X₁₀ is D or G; X₁₁ is G or H; X₁₂ is P, S, or A; X₁₃ is Y or F; X₁₄ is S or N; X₁₅ is D or E; X₁₆ is K or T; X₁₇ is I, A, or F; X₁₈ is K, R, or T; X₁₉ is D or G; X₂₀ is F or Y; X₂₁ is R or K; X₂₂ is S or T; X₂₃ is L, V, or I; and X₂₄ is L or V; X₂₅ is K or Q; X₂₆ is T or S; X₂₇ is H or Y; and X₂₈ is I or V.

6. The agent of claim 1, wherein the agent comprises an anti-TREML antibody or antigen-binding fragment thereof comprising
(i) a heavy chain variable (VH) region, wherein the VH region comprises a heavy chain complementarity determining region 1 (CDR-H1) comprising DYGMA (SEQ ID NO: 20); a CDR-H2 comprising FISNLAYRIYYADTVTG (SEQ ID NO: 27); and a CDR-H3 comprising EDYGNNGAMDY (SEQ ID NO: 28); and
a light chain variable (VL) region, wherein the VL region comprises a light chain complementarity region 1 (CDR-L1) comprising RSSQSIVHSNGNTYLE (SEQ ID NO: 26); a CDR-L2 comprising KVSNRFS (SEQ ID NO: 24); and a CDR-L3 comprising FQGSHVPPT (SEQ ID NO: 25);
(ii) a heavy chain variable (VH) region, wherein the VH region comprises a heavy chain complementarity determining region 1 (CDR-H1) comprising DYGMA (SEQ ID NO: 20); a CDR-H2 comprising FISNLAYSVYYADTVTG (SEQ ID NO: 21); and a CDR-H3 comprising EDYGDNGAIDY (SEQ ID NO: 22); and
a light chain variable (VL) region, wherein the VL region comprises a light chain complementarity region 1 (CDR-L1) comprising RSSQNIVHSNGNTYLE (SEQ ID NO: 23); a CDR-L2 comprising KVSNRFS (SEQ ID NO: 24); and a CDR-L3 comprising FQGSHVPPT (SEQ ID NO: 25);
(iii) a heavy chain variable (VH) region, wherein the VH region comprises a heavy chain complementarity determining region 1 (CDR-H1) comprising DYGMA (SEQ ID NO: 20); a CDR-H2 comprising FISNLAYSVYYADTVTG (SEQ ID NO: 21); and a CDR-H3 comprising EDYGDNGAIDY (SEQ ID NO: 22); and
a light chain variable (VL) region, wherein the VL region comprises a light chain complementarity region 1 (CDR-L1) comprising RSSQSIVHSNGNTYLE (SEQ ID NO: 26); a CDR-L2 comprising KVSNRFS (SEQ ID NO: 24); and a CDR-L3 comprising FQGSHVPPT (SEQ ID NO: 25);
(iv) a heavy chain variable (VH) region, wherein the VH region comprises a CDR-H1 comprising DYVIH (SEQ ID NO: 32); a CDR-H2 comprising YMNPYTDGPKYSDKIKD (SEQ ID NO: 33); and a CDR-H3 comprising DFNYYVGAMDF (SEQ ID NO: 34); and
a light chain variable (VL) region, wherein the VL region comprises a CDR-L1 comprising RSSQSLLHSNGNTYLH (SEQ ID NO: 35); a CDR-L2 comprising KVSNRFS (SEQ ID NO: 24); and a CDR-L3 comprising SQSTHIWT (SEQ ID NO: 36);
(v) a heavy chain variable (VH) region, wherein the VH region comprises a CDR-H1 comprising DYVIH (SEQ ID NO: 32); a CDR-H2 comprising YINPYTGGPKYSETARG (SEQ ID NO: 37); and a CDR-H3 comprising DFNYYVGAMDF (SEQ ID NO: 34); and
a light chain variable (VL) region, wherein the VL region comprises a CDR-L1 comprising RSTQSLVHSNGNTYVH (SEQ ID NO: 38); a CDR-L2 comprising KVSNRFS (SEQ ID NO: 24); and a CDR-L3 comprising SQSTYVWT (SEQ ID NO: 56);
(vi) a heavy chain variable (VH) region, wherein the VH region comprises a CDR-H1 comprising EYVIH (SEQ ID NO: 39); a CDR-H2 comprising YFNPYTGGSKFNEKFKD (SEQ ID NO: 40); and a CDR-H3 comprising DFNYYVGAMDY (SEQ ID NO: 55); and
a light chain variable (VL) region, wherein the VL region comprises a CDR-L1 comprising RSSQSLVHSNGNTYLH (SEQ ID NO: 41); a CDR-L2 comprising KVSNRFS (SEQ ID NO: 24); and a CDR-L3 comprising SQSSHIWT (SEQ ID NO: 42);
(vii) a heavy chain variable (VH) region, wherein the VH region comprises a CDR-H1 comprising NYVMH (SEQ ID NO: 43); a CDR-H2 comprising YFNPYNGHAKYSEKFTG (SEQ ID NO: 44); and a CDR-H3 comprising DFNYYVGAMDY (SEQ ID NO: 55); and
a light chain variable (VL) region, wherein the VL region comprises a CDR-L1 comprising RSSQSLIHSNGNTYLH (SEQ ID NO: 45); a CDR-L2 comprising QVSKRFS (SEQ ID NO: 46); and a CDR-L3 comprising SQSTHIWT (SEQ ID NO: 36); or
(viii) a heavy chain variable (VH) region, wherein the VH region comprises a CDR-H1 comprising DYVIH (SEQ ID NO: 32); a CDR-H2 comprising YINPYTGGPKYSETAKG (SEQ ID NO: 47); and a CDR-H3 comprising DFNYYVGAMDF (SEQ ID NO: 34); and
a light chain variable (VL) region, wherein the VL region comprises a CDR-L1 comprising KSTQSLVHSNGNTYVH (SEQ ID NO: 48); a CDR-L2 comprising KVSNRFS (SEQ ID NO: 24); and a CDR-L3 comprising SQSTYVWT (SEQ ID NO: 56).

7. The agent of claim 1, wherein the agent comprises
(i) a VH region comprising SEQ ID NO: 4 and a VL region comprising SEQ ID NO: 5;
(ii) a VH region comprising SEQ ID NO: 6 and a VL region comprising SEQ ID NO: 7;
(iii) a VH region comprising SEQ ID NO: 8 and a VL region comprising SEQ ID NO: 9;
(iv) a VH region comprising SEQ ID NO: 10 and a VL region comprising SEQ ID NO: 11;
(v) a VH region comprising SEQ ID NO: 12 and a VL region comprising SEQ ID NO: 13;
(vi) a VH region comprising SEQ ID NO: 14 and a VL region comprising SEQ ID NO: 15;
(vii) a VH region comprising SEQ ID NO: 16 and a VL region comprising SEQ ID NO: 17; or
(viii) a VH region comprising SEQ ID NO: 18 and a VL region comprising SEQ ID NO: 19.

8. A method of treating or alleviating one or more symptoms of a disease or condition associated or characterized by immune suppression in a subject in need thereof, the method comprising administering an agent of-any claim 1 to the subject, and reversing immune suppression in the subject, thereby treating or alleviating one or more symptoms of the disease or condition.

9. The method of claim 8, wherein reversing immune suppression comprises inhibiting an inflammation.

10. The method of claim 8, wherein the inflammatory response can be a monocyte-mediated, macrophage-mediated, and/or neutrophil-mediated inflammatory response.

11. The method of claim 8, wherein the disease or condition comprises malignant tumor growth, tumor angiogenesis, cancer, chronic infection, sepsis, immune exhaustion, or immunosenescence in aging.

12. The method of claim 11, wherein the cancer comprises melanoma, lung cancer, squamous cell carcinomas of the lung, head and neck cancer, breast cancer, ovarian cancer, uterine cancer, prostate cancer, gastric carcinoma, cervical cancer, esophageal carcinoma, bladder cancer, kidney cancer, brain cancer, liver cancer, colon cancer, bone cancer, pancreatic cancer, skin cancer, cutaneous or intraocular malignant melanoma, ovarian cancer, rectal cancer, cancer of the anal region, stomach cancer, testicular cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, esophagus cancer, small intestine cancer, endocrine system cancer, thyroid gland cancer, parathyroid gland cancer, adrenal gland cancer, sarcoma of soft tissue, urethra cancer, penis cancer, chronic or acute leukemia, solid tumors of childhood, lymphocytic lymphoma, carcinoma of the renal pelvis, neoplasm of the central nervous system (CNS), primary CNS lymphoma, spinal axis tumor, brain stem glioma, pituitary adenoma, Kaposi's sarcoma, epidermoid cancer, squamous cell cancer, T cell lymphoma, and gastrointestinal tract cancer, optionally wherein the cancer comprises colon cancer.

13. The method of claim 12, wherein the chronic or acute leukemia is acute myeloid leukemia, chronic myeloid leukemia, acute lymphoblastic leukemia, or chronic lymphocytic leukemia.

14. A method of inhibiting the binding of TREML1 to CD11b, wherein the method comprises administering the agent of claim 1 to cells expressing CD11 b and are in the presence of TREML1, thereby inhibiting the binding of TREML1 to CD11b.

15. A method of reversing immune suppression in cells, wherein the method comprises administering the agent of claim 1 to cells induced by TREML1 binding I-domain of CD11 b to be immune suppressive.

16. A method of inhibiting PD-L1 expression on cells, wherein the method comprises administering the agent of claim 1 to cells induced by TREML1 binding I-domain of CD11 b to express PD-L1.

17. The method of claim 14, wherein the cells comprise immune cells, and optionally wherein the immune cells are immune cells of innate immune response system.

18. (canceled)

19. The method of claim 18, wherein the cells comprise macrophages, neutrophils, monocytes, dendritic cells, natural killer cells, and granulocytes.

20. The method of claim 14, wherein the cells are in a subject, and optionally wherein the subject is a mammal.

21. (canceled)

22. A method of diagnosing cancer or detecting the presence of a malignant tumor in a subject and/or determining whether a subject would be responsive to one or more cancer therapies, wherein the method comprises obtaining a biological sample from the subject, and detecting TREML1 in the sample, thereby detecting cancer or tumor in the subject.

23.-29. (canceled)


SEQUENCE LISTING (56 sequences)

SEQ ID NO: 1 - protein, 233 aa, synthetic construct
MRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSHHHHHH

SEQ ID NO: 2 - protein, 154 aa, Homo sapiens
MHHHHHHQGIVGSLPEVLQAPVGSSILVQCHYRLQDVKAQKVWCRFLPEGCQPLVSSAVDRRAPAGRRTFLTDLGGGLLQVEMVTLQEEDAGEYGCMVDGARGPQILHRVSLNILPPEEEEETHKIGSLAENAFSDPAGSANPLEPSQDEKSIP

SEQ ID NO: 3 - protein, 162 aa, Mus musculus
MHHHHHHDSHPEVLQAPVGSSILVQCHYRLQDVRALKVWCQFLQEGCHPLVTSAVDRRAPGNGRIFLTDLGGGLLQVEMVTLQEEDTGEYGCVVEGAAGPQTLHRVSLLVLPPVPGPREGEEAEDEKETYRIGTGSLLEDPSLDPSASAGPHEFRRRENSIP

SEQ ID NO: 4 - protein, 120 aa, synthetic construct
EVKLVESGGGLVQPGGSRKLSCAASGFTFSDYGMAWVRQAPGKGPEWIAFISNLAYSVYYADTVTGRFTISRENAKNTLYLEMSSLRSEDTAMYYCAREDYGDNGAIDYWGQGTSVTVSS

SEQ ID NO: 5 - protein, 112 aa, synthetic construct
DVLMTQTPLSLPVSLGDQASISCRSSQNIVHSNGNTYLEWYLQKPGQSPQLLIYKVSNRFSGVPDRFSGSGSGTDFTLKISRVEAEDLGVYYCFQGSHVPPTFGGGTKLEIK

SEQ ID NO: 6 - protein, 120 aa, synthetic construct; identical to SEQ ID NO: 4

SEQ ID NO: 7 - protein, 112 aa, synthetic construct
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPPTFGGGTKLEIK

SEQ ID NO: 8 - protein, 120 aa, synthetic construct
EVKLVESGGGLVQPGGSRKLSCAASGFTFSDYGMAWVRQAPGKGPEWIAFISNLAYRIYYADTVTGRFTISRENAKNTLYLEMSSLRSEDSAIYYCVREDYGNNGAMDYWGQGTSVTVSS

SEQ ID NO: 9 - protein, 112 aa, synthetic construct; identical to SEQ ID NO: 7

SEQ ID NO: 10 - protein, 120 aa, synthetic construct
EVQVQQSGPELVNPGASVRLSCKASGYIFTDYVIHWVRQKPGQGLEWIGYMNPYTDGPKYSDKIKDKAILTSDRSSNTVYMELRSLTSEDSAVYFCARDFNYYVGAMDFWGQGTSVTVSS

SEQ ID NO: 11 - protein, 111 aa, synthetic construct
DVVLTQTPLSLPVSLGDQASVSCRSSQSLLHSNGNTYLHWYLQKPGQSPKLLIYKVSNRFSGVPDRFTGSGSGTDFTLKISRVEAEDLGVYFCSQSTHIWTFGGGTKLEIK

SEQ ID NO: 12 - protein, 120 aa, synthetic construct
EVQLQQSGPELVRPGASVKVSCKASGYIFTDYVIHWVKQKPGQGLEWFGYINPYTGGPKYSETARGKATLTSDSSSNTAYMVLTSLTSDDSAVYYCTRDFNYYVGAMDFWGQGTSVTVSS

SEQ ID NO: 13 - protein, 111 aa, synthetic construct
AAVLTQTPLSLPVSLGDQASISCRSTQSLVHSNGNTYVHWYLQKAGQSPKLLIYKVSNRFSGVPDRFSGSGSGTDFTLKISRVEAEDLGVYFCSQSTYVWTFGGGTKLEIE

SEQ ID NO: 14 - protein, 120 aa, synthetic construct
DVQLQQSGPELVKPGASVKMSCKASGYTFTEYVIHWVKQRPGQGLEWFGYFNPYTGGSKFNEKFKDKATLTSDRSSNTVYMELYSLTSEDSAVYYCARDFNYYVGAMDYWGQGTSVTVSS

SEQ ID NO: 15 - protein, 111 aa, synthetic construct
DVALTQTPLSLPVSLGDQASISCRSSQSLVHSNGNTYLHWYLQKSGQSPKLLIYKVSNRFSGVPDRFSGSGSVTEFTLKISRVEAEDLGVYFCSQSSHIWTFGGGTRLEIK

SEQ ID NO: 16 - protein, 120 aa, synthetic construct
EVQLQQSGPELVKPGASVKMSCKASGYTFTNYVMHWVKQKPGQGLEWIGYFNPYNGHAKYSEKFTGKATLTSDRSSSTAYMELNSLTSEDSAVYYCTRDFNYYVGAMDYWGQGTSVTVSS

SEQ ID NO: 17 - protein, 111 aa, synthetic construct
DVVMTQSPLSLPVNLGDHASISCRSSQSLIHSNGNTYLHWYLQKPGQSPKLLVYQVSKRFSGVPDRFSGSGSGTDFTLKISRVEAEDLGVYFCSQSTHIWTFGGGTTLEIK

SEQ ID NO: 18 - protein, 120 aa, synthetic construct
EVQLQQSGPELVRPGASVKVSCKASGYIFTDYVIHWVKQKPGQGLEWFGYINPYTGGPKYSETAKGKATLTSDSSSNTAYMVLTSLTSEDSAVYYCTRDFNYYVGAMDFWGQGTSVTVSS

SEQ ID NO: 19 - protein, 111 aa, synthetic construct
GAVLTQTPLSLPVSLGDQASISCKSTQSLVHSNGNTYVHWYLQKAGQSPKLLIYKVSNRFSGVPDRFIGSGSGTDFTLKISRVEAEDLGVYFCSQSTYVWTFGGGTKLEIE

SEQ ID NO: 20 - protein, 5 aa, synthetic construct
DYGMA

SEQ ID NO: 21 - protein, 17 aa, synthetic construct
FISNLAYSVYYADTVTG

SEQ ID NO: 22 - protein, 11 aa, synthetic construct
EDYGDNGAIDY

SEQ ID NO: 23 - protein, 16 aa, synthetic construct
RSSQNIVHSNGNTYLE

SEQ ID NO: 24 - protein, 7 aa, synthetic construct
KVSNRFS

SEQ ID NO: 25 - protein, 9 aa, synthetic construct
FQGSHVPPT

SEQ ID NO: 26 - protein, 16 aa, synthetic construct
RSSQSIVHSNGNTYLE

SEQ ID NO: 27 - protein, 17 aa, synthetic construct
FISNLAYRIYYADTVTG

SEQ ID NO: 28 - protein, 11 aa, synthetic construct
EDYGNNGAMDY

SEQ ID NO: 29 - protein, 17 aa, synthetic construct
FISNLAYXXYYADTVTG

SEQ ID NO: 30 - protein, 11 aa, synthetic construct
EDYGXNGAXDY

SEQ ID NO: 31 - protein, 16 aa, synthetic construct
RSSQXIVHSNGNTYLE

SEQ ID NO: 32 - protein, 5 aa, synthetic construct
DYVIH

SEQ ID NO: 33 - protein, 17 aa, synthetic construct
YMNPYTDGPKYSDKIKD

SEQ ID NO: 34 - protein, 11 aa, synthetic construct
DFNYYVGAMDF

SEQ ID NO: 35 - protein, 16 aa, synthetic construct
RSSQSLLHSNGNTYLH

SEQ ID NO: 36 - protein, 8 aa, synthetic construct
SQSTHIWT

SEQ ID NO: 37 - protein, 17 aa, synthetic construct
YINPYTGGPKYSETARG

SEQ ID NO: 38 - protein, 16 aa, synthetic construct
RSTQSLVHSNGNTYVH

SEQ ID NO: 39 - protein, 5 aa, synthetic construct
EYVIH

SEQ ID NO: 40 - protein, 17 aa, synthetic construct
YFNPYTGGSKFNEKFKD

SEQ ID NO: 41 - protein, 16 aa, synthetic construct
RSSQSLVHSNGNTYLH

SEQ ID NO: 42 - protein, 8 aa, synthetic construct
SQSSHIWT

SEQ ID NO: 43 - protein, 5 aa, synthetic construct
NYVMH

SEQ ID NO: 44 - protein, 17 aa, synthetic construct
YFNPYNGHAKYSEKFTG

SEQ ID NO: 45 - protein, 16 aa, synthetic construct
RSSQSLIHSNGNTYLH

SEQ ID NO: 46 - protein, 7 aa, synthetic construct
QVSKRFS

SEQ ID NO: 47 - protein, 17 aa, synthetic construct
YINPYTGGPKYSETAKG

SEQ ID NO: 48 - protein, 16 aa, synthetic construct
KSTQSLVHSNGNTYVH

SEQ ID NO: 49 - skipped

SEQ ID NO: 50 - protein, 17 aa, synthetic construct
YXNPYXXXXKXXXXXXX

SEQ ID NO: 51 - protein, 11 aa, synthetic construct
DFNYYVGAMDX

SEQ ID NO: 52 - protein, 16 aa, synthetic construct
XSXQSLXHSNGNTYXH

SEQ ID NO: 53 - protein, 7 aa, synthetic construct
XVSNRFS

SEQ ID NO: 54 - protein, 8 aa, synthetic construct
SQSXXXWT

SEQ ID NO: 55 - protein, 11 aa, synthetic construct
DFNYYVGAMDY

SEQ ID NO: 56 - protein, 8 aa, synthetic construct
SQSTYVWT